SEQUENCE LISTING (2 sequences)

SEQ ID NO: 1 - DNA, 2148 bp, Escherichia coli BW25113
atgaacgttattgcaatattgaatcacatgggggtttattttaaagaagaacccatccgtgaacttcatcgcgcgcttgaacgtctgaacttccagattgtttacccgaacgaccgtgacgacttattaaaactgatcgaaaacaatgcgcgtctgtgcggcgttatttttgactgggataaatataatctcgagctgtgcgaagaaattagcaaaatgaacgagaacctgccgttgtacgcgttcgctaatacgtattccactctcgatgtaagcctgaatgacctgcgtttacagattagcttctttgaatatgcgctgggtgctgctgaagatattgctaataagatcaagcagaccactgacgaatatatcaacactattctgcctccgctgactaaagcactgtttaaatatgttcgtgaaggtaaatatactttctgtactcctggtcacatgggcggtactgcattccagaaaagcccggtaggtagcctgttctatgatttctttggtccgaataccatgaaatctgatatttccatttcagtatctgaactgggttctctgctggatcacagtggtccacacaaagaagcagaacagtatatcgctcgcgtctttaacgcagaccgcagctacatggtgaccaacggtacttccactgcgaacaaaattgttggtatgtactctgctccagcaggcagcaccattctgattgaccgtaactgccacaaatcgctgacccacctgatgatgatgagcgatgttacgccaatctatttccgcccgacccgtaacgcttacggtattcttggtggtatcccacagagtgaattccagcacgctaccattgctaagcgcgtgaaagaaacaccaaacgcaacctggccggtacatgctgtaattaccaactctacctatgatggtctgctgtacaacaccgacttcatcaagaaaacactggatgtgaaatccatccactttgactccgcgtgggtgccttacaccaacttctcaccgatttacgaaggtaaatgcggtatgagcggtggccgtgtagaagggaaagtgatttacgaaacccagtccactcacaaactgctggcggcgttctctcaggcttccatgatccacgttaaaggtgacgtaaacgaagaaacctttaacgaagcctacatgatgcacaccaccacttctccgcactacggtatcgtggcgtccactgaaaccgctgcggcgatgatgaaaggcaatgcaggtaagcgtctgatcaacggttctattgaacgtgcgatcaaattccgtaaagagatcaaacgtctgagaacggaatctgatggctggttctttgatgtatggcagccggatcatatcgatacgactgaatgctggccgctgcgttctgacagcacctggcacggcttcaaaaacatcgataacgagcacatgtatcttgacccgatcaaagtcaccctgctgactccggggatggaaaaagacggcaccatgagcgactttggtattccggccagcatcgtggcgaaatacctcgacgaacatggcatcgttgttgagaaaaccggtccgtataacctgctgttcctgttcagcatcggtatcgataagaccaaagcactgagcctgctgcgtgctctgactgactttaaacgtgcgttcgacctgaacctgcgtgtgaaaaacatgctgccgtctctgtatcgtgaagatcctgaattctatgaaaacatgcgtattcaggaactggctcagaatatccacaaactgattgttcaccacaatctgccggatctgatgtatcgcgcatttgaagtgctgccgacgatggtaatgactccgtatgctgcattccagaaagagctgcacggtatgaccgaagaagtttacctcgacgaaatggtaggtcgtattaacgccaatatgatccttccgtacccgccgggagttcctctggtaatgccgggtgaaatgatcaccgaagaaagccgtccggttctggagttcctgcagatgctgtgtgaaatcggcgctcactatccgggctttgaaaccgatattcacggtgcataccgtcaggctgatggccgctataccgttaaggtattgaaagaagaaagcaaaaaataa

SEQ ID NO: 2 - protein, 715 aa, Escherichia coli BW25113
MNVIAILNHMGVYFKEEPIRELHRALERLNFQIVYPNDRDDLLKLIENNARLCGVIFDWDKYNLELCEEISKMNENLPLYAFANTYSTLDVSLNDLRLQISFFEYALGAAEDIANKIKQTTDEYINTILPPLTKALFKYVREGKYTFCTPGHMGGTAFQKSPVGSLFYDFFGPNTMKSDISISVSELGSLLDHSGPHKEAEQYIARVFNADRSYMVTNGTSTANKIVGMYSAPAGSTILIDRNCHKSLTHLMMMSDVTPIYFRPTRNAYGILGGIPQSEFQHATIAKRVKETPNATWPVHAVITNSTYDGLLYNTDFIKKTLDVKSIHFDSAWVPYTNFSPIYEGKCGMSGGRVEGKVIYETQSTHKLLAAFSQASMIHVKGDVNEETFNEAYMMHTTTSPHYGIVASTETAAAMMKGNAGKRLINGSIERAIKFRKEIKRLRTESDGWFFDVWQPDHIDTTECWPLRSDSTWHGFKNIDNEHMYLDPIKVTLLTPGMEKDGTMSDFGIPASIVAKYLDEHGIVVEKTGPYNLLFLFSIGIDKTKALSLLRALTDFKRAFDLNLRVKNMLPSLYREDPEFYENMRIQELAQNIHKLIVHHNLPDLMYRAFEVLPTMVMTPYAAFQKELHGMTEEVYLDEMVGRINANMILPYPPGVPLVMPGEMITEESRPVLEFLQMLCEIGAHYPGFETDIHGAYRQADGRYTVKVLKEESKK

(19) United States
(12) Patent Application Publication
(10) Pub. No.: US 2023/0110819 A1
(43) Pub. Date: Apr. 13, 2023

(54) BIO-BASED NYLON PRECURSORS HAVING REDUCED ORGANIC AND INORGANIC IMPURITIES

(71) Applicant: MOJIA BIOTECH LTD., Shanghai (CN)

(72) Inventors: Ansen Chiew, Shanghai (CN); Man Kit Lau, Shanghai (CN); Congming Zeng, Shanghai (CN); Tailong Jiang, Shanghai (CN); Jinhuan Su, Shanghai (CN); Shangtong Yang, Shanghai (CN); Chengliang Lu, Shanghai (CN)

(21) Appl. No.: 17/911,409

(22) PCT Filed: Mar. 14, 2021
(86) PCT No.: PCT/CN2021/080654

§ 371 Date: Sep. 13, 2022

(30) Foreign Application Priority Data
Mar. 14, 2020 (CN) PCT/CN2020/079405

(51) Int. Cl. C12P 13/02; C08G 69/28; C12N 9/88

(52) U.S. Cl. CPC C12P 13/02; C08G 69/28; C12N 9/88; C12Y 401/01018

(57) ABSTRACT

Improved processes for producing bio-based nylon precursors having reduced organic and inorganic impurities are described herein. The processes generally comprise fermenting a microorganism engineered to produce lysine in a modified culture medium having low or reduced inorganic ion content, such as by employing a culture medium having an ammonium dicarboxylate buffering system that is preferably devoid of non-essential inorganic ions, and crystallizing the lysine directly from the spent lysine fermentation supernatant by adding a sufficient amount of a dicarboxylic acid. Such strategies aim to produce lysine dicarboxylate salt crystals that are employable in a downstream bioconversion step for the production of cadaverine dicarboxylate salts having reduced organic and inorganic impurities, which improve their downstream performance, for example in polymeration reactions for polyamide synthesis.

The present description relates to bio-based nylon precursors. More particularly, described herein are improved processes for the production of bio-based nylon precursors, such as lysine, cadaverine, and dicarboxylate salts thereof, having reduced organic and inorganic impurities suitable for use in the synthesis of nylons.

### BACKGROUND

Growing global environmental concerns have led to an increasing demand for polyamides derived from biological precursors, commonly known as bio-based nylons. Most nylons are polymers made from the reaction of a diamine with a dicarboxylic acid. Bio-based nylons, such as bio-based nylon 6,6 and the like, have become increasingly important industrial materials. While bio-based nylon 5,6 is attracting more and more attention, one of the challenges it faces is the purity requirement, because impurities in the nylon 5,6 product may cause yellowing, intolerance to high temperature and other adverse properties in subsequent applications. An important building block of bio-based nylon 5,6 is the five-carbon diamine cadaverine (pentamethylenediamine; PDA), which can be produced industrially for example by fermenting microorganisms specifically engineered for this purpose, or by bioconversion involving enzymatic decarboxylation of L-lysine (Tsuge et al., 2016). While the former suffers from low productivity due to detrimental effects of cadaverine on the host microorganism, the latter is difficult to implement viably at an industrial scale due to the high cost and low purity of the substrate L-lysine. It has been found that in the fermentative production process of cadaverine or fermentative production process of lysine, due to the substrate, medium, pH adjustment process, etc., a large amount of inorganic salts will inevitably be produced, and these inorganic salts require complicated processing steps to remove. There are also studies in the prior art focusing on reducing the inorganic salts formed in the fermentation production process of cadaverine or the fermentation production process of lysine. However, when cadaverine is produced by fermentation, it is conventional in the field of biological fermentation to favor a “one-pot” continuous process that minimizes mid-process purification steps (e.g., of intermediates) to improve overall efficiency and reduce operating costs, with expensive purification steps generally being reserved for the final products. There thus remains a need for technological advancements that would better enable bio-based nylons to compete on an industrial scale with their petroleum-based counterparts. In particular, there remains a need for technological advancements in the production of bio-based nylon and its precursors in a very efficient and high-purity manner.

### SUMMARY

The inventors have unexpectedly discovered that by using the processes disclosed herein, cadaverine dicarboxylate salts can be obtained in high yield and purity, and nylon 5,X polymers prepared from such cadaverine dicarboxylate salts are of high quality, for example, no yellowing or intolerance to high temperature will occur in use.

In one aspect, described herein is a process for producing a cadaverine dicarboxylate salt having reduced inorganic ion content, the process comprising or consisting essentially of: (a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions; (b) fermenting the microorganism in the presence of the carbon source under culture conditions enabling lysine production, while controlling fermentation broth pH with the addition of ammonium hydroxide to maintain pH in a range conducive to lysine production; (c) adding less than one equivalent or excess dicarboxylic acid to obtain lysine dicarboxylate salt crystals and dissolving the crystals in aqueous solution, to obtain a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system; (d) subjecting the lysine dicarboxylate salt in the lysine dicarboxylate salt stream to an enzymatic decarboxylation reaction while maintaining the pH of the solution at a level sufficient for said reaction to occur by adding the ammonium dicarboxylate buffering system to said solution, thereby producing a solution comprising cadaverine dicarboxylate; and (e) crystallizing cadaverine dicarboxylate salt by adding a sufficient volume of an organic solvent to the solution.

In further aspects, described herein is a process for producing a cadaverine dicarboxylate salt having reduced inorganic ion content, the process comprising or consisting essentially of: (a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions; (b) fermenting the microorganism in the presence of the carbon source under culture conditions enabling lysine production, while controlling fermentation broth pH with the addition of ammonium hydroxide to maintain pH in a range conducive to lysine production; (c) obtaining a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system; (d) subjecting the lysine dicarboxylate salt in the lysine dicarboxylate salt stream to an enzymatic decarboxylation reaction while maintaining the pH of the lysine dicarboxylate salt stream at a level sufficient for said reaction to occur by adding the ammonium dicarboxylate buffering system to said stream, thereby producing a solution comprising cadaverine dicarboxylate; and (e) crystallizing cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol, and being especially preferably isopropanol.

In further aspects, described herein is a process for producing a cadaverine dicarboxylate salt having reduced inorganic ion content, the process comprising or consisting essentially of: (a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions; (b) fermenting the microorganism in the presence of the carbon source under culture conditions enabling lysine production, while controlling fermentation broth pH with the addition of ammonium hydroxide to maintain pH in a range conducive to lysine production; (c) obtaining a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system; (d) subjecting the lysine dicarboxylate salt in the lysine dicarboxylate salt stream to an enzymatic decarboxylation reaction while maintaining the pH of the lysine dicarboxylate salt stream at a level sufficient for said reaction to occur by adding the ammonium dicarboxylate buffering system and adding a high activity lysine decarboxylase described herein in a significantly reduced lysine decarboxylase addition ratio compared to the prior art, thereby producing a solution comprising cadaverine dicarboxylate; and (e) crystallizing cadaverine dicarboxylate salt by adding a sufficient volume of an organic solvent to the solution.

The high activity lysine decarboxylase described herein can be used in a significantly reduced amount during the fermentation process due to its relatively higher activity. In addition, the inventors have discovered that by using the high activity lysine decarboxylase described herein, the yield and purity of the target product cadaverine dicarboxylate salt can also be improved. The high activity lysine decarboxylase described herein is the lysine decarboxylase which is treated by the process comprising the following steps: (1) plasmid containing the kdc gene of the lysine decarboxylase represented by SEQ ID NO: 1 was transformed into E. coli cells; (2) the transformed positive single colony was selected and inoculated into a LB (10 g/L peptone, 5 g/L yeast extract, and 10 g/L sodium chloride) test tube medium, and the medium was inoculated at 30° C. and 180 RPM overnight; (3) flasks containing TB medium (12 g/L peptone, 24 g/L yeast extract, and 4 g/L glycerol) were inoculated at 5% inoculation dosage from the overnight cultures; (4) the flasks were placed into a shaker for incubation, incubation condition: 30° C., 250 RPM, and for about 2 hours; (5) protein expression was induced with 0.2 mM Isopropyl β-D-1-thiogalactopyranoside (IPTG), the induction condition: 30° C., 250 RPM, and for about 4 hours; (6) cells were harvested by centrifugating the fermentation broth and the wet cells after centrifugation were stored at −80° C.

In further aspects, described herein is a process for producing a cadaverine dicarboxylate salt having reduced inorganic ion content, the process comprising or consisting essentially of: (a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions: (b) fermenting the microorganism in the presence of the carbon source under culture conditions enabling lysine production, while controlling fermentation broth pH with the addition of ammonium hydroxide to maintain pH in a range conducive to lysine production: (c) adding less than one equivalent or excess dicarboxylic acid to obtain lysine dicarboxylate salt crystals and dissolving the crystals in aqueous solution, to obtain a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system; (d) subjecting the lysine dicarboxylate salt in the lysine dicarboxylate salt stream to an enzymatic decarboxylation reaction while maintaining the pH of the solution at a level sufficient for said reaction to occur by adding the ammonium dicarboxylate buffering system and adding a high activity lysine decarboxylase described herein in a significantly reduced lysine decarboxylase addition ratio compared to the prior art, thereby producing a solution comprising cadaverine dicarboxylate; and (e) crystallizing cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol, and being especially preferably isopropanol.

In further aspects, described herein is a cadaverine dicarboxylate salt produced by a process described herein, and use of the cadaverine dicarboxylate salt produced by a process described herein for the manufacture of a nylon.

In a further aspect, described herein is a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions, wherein the fermentation broth has reduced inorganic ion content as compared to a corresponding fermentation broth employing an inorganic anion instead of a dicarboxylate anion in the buffering system.

In a further aspect, described herein is a lysine dicarboxylate salt stream obtained from a lysine fermentation, the lysine dicarboxylate salt stream comprising lysine cations, dicarboxylate anions, and culture medium modified to comprise an ammonium dicarboxylate buffering system and preferably to be devoid of non-essential inorganic ions, wherein the lysine dicarboxylate salt stream has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.

In a further aspect, described herein is a process for producing lysine dicarboxylate salt crystals having reduced inorganic impurities and improved performance in downstream enzymatic bioconversion reactions to produce cadaverine dicarboxylate salts. The process generally comprises or consists essentially of: (a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions; (b) fermenting the microorganism in the presence of the carbon source under culture conditions enabling lysine production, while controlling fermentation broth pH with the addition of ammonium hydroxide to maintain pH in a range conducive to lysine production; and (c) adding a sufficient amount of dicarboxylic acid to the spent fermentation broth to induce formation of lysine dicarboxylate salt crystals. The lysine dicarboxylate salt crystals produced have reduced inorganic ion content as compared to a lysine inorganic salt obtainable via a corresponding process in which an inorganic anion is substituted for the dicarboxylate anion in the buffering system.

In a further aspect, described herein is a process for producing a cadaverine dicarboxylate salt having reduced organic and inorganic impurities, the process comprising or consisting essentially of steps (a) to (c) as defined above, the process further comprising: (d) dissolving the isolated lysine dicarboxylate salt crystals from step (c) in aqueous solution and subjecting the lysine to an enzymatic decarboxylation reaction while maintaining the pH of the solution at a level sufficient for said reaction to occur by adding dicarboxylic acid to said solution, thereby producing a solution comprising cadaverine dicarboxylate; and (e) crystallizing cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol.

In further aspects, described herein is a lysine dicarboxylate salt or cadaverine dicarboxylate salt produced by a process described herein, and use of the lysine dicarboxylate salt or the cadaverine dicarboxylate salt produced by a process described herein for the manufacture of a nylon.

In a further aspect, described herein is a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions, wherein the fermentation broth has reduced inorganic ion content as compared to a corresponding fermentation broth employing an inorganic anion instead of a dicarboxylate anion in the buffering system.

In a further aspect, described herein is a lysine dicarboxylate salt stream obtained from a lysine fermentation, the lysine dicarboxylate salt stream comprising lysine cations, dicarboxylate anions, and culture medium modified to comprise an ammonium dicarboxylate buffering system and preferably to be devoid of non-essential inorganic ions, wherein the lysine dicarboxylate salt stream has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or process/method steps.

The expression “consisting essentially or” as used herein refers to those elements required for a given embodiment as well as insubstantial elements, and excludes elements that would materially change the invention. For example, the processes described herein generally relate to reducing or minimizing the amounts of inorganic ions (or non-dicarboxylate ions) and/or increasing the purity of the nylon precursors contemplated herein. Accordingly, the expression “consisting essentially of” in such contexts is meant to exclude steps and elements that would introduce additional unwanted inorganic ions (or non-dicarboxylate ions) and/or reduce the purity of the nylon precursors, thereby going against the teachings of the present description.

As used herein, the term “inorganic” in the context of ions/counter ions described herein refers to ions (e.g., negative ions or anions) lacking C—H bonds, such as phosphates, sulfates, and/or chlorides. Such inorganic ions are commonly used in the art of fermentation buffers and/or reaction solutions in buffering agents and/or acids/bases utilized for adjusting or controlling pH.

As used herein, the term “lysine” generally refers to L-lysine unless otherwise specified. In general, the fermentation broth in processes described herein comprises lysine predominantly in its monocationic form (Lys+) as a dicarboxylate salt, due to the pH ranges of the fermentations (e.g., pH 3-8).

### BRIEF DESCRIPTION OF THE DRAWINGS

In the appended drawings:

FIG. 1 shows the time-course of a conventional lysine fermentation process in ammonium sulfate-containing culture medium performed as described in Example 4A.

### SEQUENCE LISTING

This application contains a Sequence Listing in computer readable form created Feb. 24, 2020 having a size of about 9 kb. The computer readable form is incorporated herein by reference.

| SEQ ID NO: | Description |
|---|---|
| 1 | 2-keto-acid decarboxylase wild-type nucleic acid |
| | sequence from E. coli strain BW25113 |
| 2 | 2-keto-acid decarboxylase wild-type amino acid |
| | sequence from E. coli strain BW25113 |

### DETAILED DESCRIPTION

As a key precursor in bio-based nylon manufacturing, cadaverine (also known as pentamethylenediamine or 1,5-pentanediamine [PDA]) can be produced by enzymatic conversion of L-lysine via lysine decarboxylases. While L-lysine in its free base form is commercially obtainable in relatively small quantities, the amino acid is currently manufactured and sold at industrial quantities only as relatively impure inorganic lysine hydrochloride or lysine sulfate salts, due to the high costs of its purification as a free base. Because the bulk of lysine produced at an industrial scale via fermentation is intended for use in animal feed mixtures, the lack of purity of commercial lysine hydrochloride or lysine sulfate salts is inconsequential for this purpose. However, the presence of chloride or sulfate ions (and other organic and/or inorganic impurities) in commercially available lysine hydrochloride or lysine sulfate salts ultimately decreases the purity of the cadaverine and/or cadaverine dicarboxylate salts produced therefrom, thereby reducing performance in nylon syntheses. As a consequence, costly purification steps are conventionally employed to remove organic and inorganic impurities from bio-based nylon precursors. Thus, improved processes for producing high purity bio-based nylon precursors having reduced organic and inorganic impurities without sacrificing yield are highly desirable.

In some aspects, described herein is a process for producing lysine dicarboxylate salt crystals having reduced inorganic impurities, which is directly employable in a downstream bioconversion step for the production of cadaverine dicarboxylate salts as precursors for bio-based nylon synthesis. The process generally comprises fermenting a microorganism engineered to produce lysine from a carbon source in a modified culture medium having low or reduced inorganic ion content, as compared to standard culture media (e.g., culture media utilizing inorganic counter anions such as ammonium sulfate or ammonium chloride) conventionally employed for fermenting such engineered microorganisms. More generally, the processes described herein relate, in part, to strategies for reducing or minimizing inorganic anion content (and/or the content of anions other than the dicarboxylate desired in the lysine salt product) throughout, thereby reducing or minimizing the amounts of counter anion impurities that ultimately become present in the intermediates and/or products produced (e.g., lysine dicarboxylate salts, cadaverine dicarboxylate salts, and nylons produced therefrom). More specifically, the processes described herein replace inorganic ammonium salts (e.g., ammonium sulfate or ammonium chloride) employed in conventional methods with an ammonium dicarboxylate buffering system. Such strategies bring down production costs by reducing or avoiding downstream purification steps to remove undesirable inorganic ions (and/or non-dicarboxylate ions) such as by ion exchange and/or distillation methods, and may provide higher purity products having improved performance in nylon synthesis reactions.

In some embodiments, the lysine fermentation processes described herein comprise a further step of crystallizing the lysine as a lysine dicarboxylate salt by adding a sufficient amount of a corresponding dicarboxylic acid directly to the spent fermentation broth/supernatant, following completion of fermentation. Strikingly, the yields/purities of the lysine salt crystals obtained using the dicarboxylic acids were either comparable to, or higher than, the yields/purities obtained using the inorganic anions/acids (e.g., sulfate/sulfuric or chloride/hydrochloric acid), despite the latter methods including an extra desalting step by passing through ion exchange columns. In some embodiments, the amount of dicarboxylic acid added to crystallize the lysine dicarboxylate salt may be at least one equivalent with respect to the mols of lysine in the spent fermentation broth. In some embodiments, the amount of dicarboxylic acid added to crystallize the lysine dicarboxylate salt may be an excess equivalent with respect to the mols of lysine in the spent fermentation broth. Interestingly, inducing lysine salt crystallization using excess dicarboxylic acid was found to be more conducive to the formation of crystals, thus resulting in increased yield, compared to using an equal equivalent amount of the dicarboxylic acid. In particular, crystallization of lysine dicarboxylate salt with excess adipic acid was found to provided the highest combination of both yield and purity amongst the different conditions tested herein.

As used herein, the expressions “lysine dicarboxylate salt”, “lysine dicarboxylate salt crystals” and “lysine dicarboxylate salt stream,” when used in the context of processes described herein, refer to a downstream product of the lysine fermentation processes described herein which has reduced inorganic ion content, as compared to lysine salt stream obtained from a corresponding lysine fermentation process in which an inorganic anion (e.g., sulfate, phosphate, or chloride) is substituted for the dicarboxylate anion in the dicarboxylate buffering system. For greater clarity, the lysine dicarboxylate salts and lysine dicarboxylate salt stream described herein includes compositions obtained downstream of the lysine fermentation that may be subjected to centrifugation and/or filtration steps to remove cells and/or cellular debris, and/or that may be subjected to a crystallization step to obtain lysine dicarboxylate salt crystals. However, the lysine dicarboxylate salt stream described herein preferably excludes any additional purification steps aimed at removing inorganic ions, such as ion exchange and/or other desalting methods conventionally employed for this purpose.

In some aspects, described herein is a process for producing a lysine dicarboxylate salt, lysine dicarboxylate salt crystals, and a lysine dicarboxylate salt stream, having reduced inorganic ion content (and/or reduced non-dicarboxylate ion content), the process comprising or consisting essentially of providing a fermentation broth comprising microorganisms immersed in a modified culture medium, the microorganisms being engineered to produce lysine from a carbon source and the culture medium being modified to comprise a dicarboxylate buffering system (e.g., an ammonium dicarboxylate buffering system such as an ammonium adipate buffering system), for example instead of a culture medium or buffering system employing inorganic counter anions (e.g., phosphate, sulfate, and/or chloride ions) or non-dicarboxylate counter anions (e.g., carbonate/bicarbonate). The process further comprises fermenting the microorganism in the presence of the carbon source under culture conditions enabling lysine production, while controlling fermentation broth pH with the addition of a nitrogen source (e.g., selected from ammonium hydroxide and ammonium dicarboxylate) to maintain pH in a range conducive to lysine production. The process further comprises obtaining lysine dicarboxylate salt crystals and/or a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system.

As used herein, the expression “non-dicarboxylate Ion” or “non-dicarboxylate anion” refers to an anion of a dicarboxylic acid other than the dicarboxylate anion employed in the ammonium dicarboxylate buffering system described herein, or other than the dicarboxylate anion present in the lysine dicarboxylate salt or cadaverine dicarboxylate salt produced by the processes described herein. Preferably, a single type of non-essential dicarboxylate anion (e.g., ammonium adipate) and corresponding dicarboxylic acid (e.g., adipic acid) is employed throughout the lysine fermentation and cadaverine dicarboxylate production processes described herein, thereby providing greater homogeneity in the dicarboxylate salts produced therefrom. For greater clarity, unless otherwise specified, the expressions “dicarboxylate” and “dicarboxylic acid” used herein refer to the species of dicarboxylate anion and corresponding dicarboxylic acid that is intended to be incorporated into the dicarboxylate salts (e.g., lysine dicarboxylate salt and/or cadaverine dicarboxylate salt) produced by the processes described herein.

In some embodiments, the modified culture medium employed in the processes described herein are preferably formulated to be devoid of non-essential inorganic counter ions (and/or non-essential non-dicarboxylate anions). As used herein, the expression “non-essential” in the context of inorganic and/or non-dicarboxylate counter anions refers to reducing the amounts of such anion (e.g., phosphate, sulfate, chloride, and/or carbonate/bicarbonate ions) in a given culture medium or fermentation broth such that the engineered microorganism being cultured/fermented is able to satisfactorily perform its desired function in the fermentation process, but excess inorganic and/or non-dicarboxylate counter anions are removed. Such modified culture media may be referred to as “minimal inorganic anion media” or “minimal non-dicarboxylate anion media” and may be tailored to different microorganisms and/or fermentation conditions, as well as to the goal of the fermentation (i.e., cell growth versus lysine production). For example, for microorganisms (e.g., stationary phase microorganisms) being fermented for the production of lysine, non-essential inorganic and/or non-dicarboxylate counter anions may refer to such anions present in the culture medium or fermentation broth that are either not required for this purpose or are present in amounts in excess of those minimally required for lysine production. For greater clarity, the minimum essential inorganic and/or non-dicarboxylate counter anions in the context of lysine production (e.g., in a production fermentation phase) may not be the same as those essential for microorganism growth (e.g., in a growth fermentation phase). Thus, in some embodiments, a first modified culture medium devoid of non-essential inorganic and/or non-dicarboxylate counter anions for cell growth may be employed in a growth phase of fermentation, wherein the microorganisms are cultured for cell growth until a desired level of cellular biomass is reached. In some embodiments, a second modified culture medium devoid of non-essential inorganic and/or non-dicarboxylate counter anions for lysine production may be employed in a production phase of fermentation, wherein the microorganisms are cultured for lysine production instead of for cellular growth. It is understood that the types and precise concentrations of inorganic anions and/or non-dicarboxylate anions present in the minimal culture medium will vary depending on the microorganism selected for the lysine fermentation, and may be determined empirically as a function of the fermentation process conditions employed.

In some embodiments, described herein is a modified culture medium for use in an industrial process for the fermentative production of a lysine dicarboxylate salt stream using an engineered microorganism, the modified culture medium comprising a dicarboxylate buffering system (e.g., an ammonium dicarboxylate buffering system such as an ammonium adipate buffering system) and preferably being devoid of non-essential inorganic and/or non-dicarboxylate counter anions. In some embodiments, described herein is a modified fermentation broth for use in an industrial process for the fermentative production of a lysine dicarboxylate salt stream, comprising microorganisms engineered to produce lysine from a carbon source immersed in modified culture medium comprising a dicarboxylate buffering system (e.g., an ammonium dicarboxylate buffering system such as an ammonium adipate buffering system) and preferably being devoid of non-essential inorganic and/or non-dicarboxylate counter anions. In some embodiments, the fermentation broth has reduced inorganic ion content as compared to a corresponding fermentation broth employing an inorganic anion instead of a dicarboxylate anion in the buffering system.

In some embodiments, described herein is a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions. In some embodiments, the fermentation broth has reduced inorganic ion content as compared to a corresponding fermentation broth employing an inorganic anion instead of a dicarboxylate anion in the buffering system. In some embodiments, the fermentation broth may be supplemented with an ammonium dicarboxylate solution to maintain total ammonium concentration at a level conducive to lysine production. In some embodiments, the fermentation broth may have a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.

In some embodiments, described herein a lysine dicarboxylate salt stream obtained from a lysine fermentation, the lysine dicarboxylate salt stream comprising lysine cations, dicarboxylate anions, and culture medium modified to comprise an ammonium dicarboxylate buffering system and preferably to be devoid of non-essential inorganic ions, wherein the lysine dicarboxylate salt stream has a Dicarboxylate Salt Ratio (DSR) of at least 50%, 55%, 60%, 65%, 70%, 75%, or 80%, as calculated as described above.

In some embodiments, the processes described herein, in a lysine fermentation step, further comprise supplementing the fermentation broth with a nitrogen source to sufficient for lysine production by the microorganism engineered to produce lysine. In some embodiments, the processes described herein comprise supplementing the fermentation broth with a nitrogen source selected from ammonium hydroxide and ammonium dicarboxylate. In some embodiments, the only nitrogen sources supplemented in the fermentation broth after the start of fermentation are selected from ammonium hydroxide and ammonium dicarboxylate. The choice whether to supplement with ammonium hydroxide or ammonium dicarboxylate can be made based on the level of pH that is to be maintained, as the former is more basic than the latter. In some embodiments, the processes described herein comprise supplementing the fermentation broth with an ammonium hydroxide and/or ammonium dicarboxylate solution to maintain total ammonium concentration at a level conducive to lysine production. In some embodiments, the nitrogen source is supplemented when the total ammonium level is reduced to about 0.15% (w/v), and/or to maintain total ammonium concentration at a level at about 0.05% to 0.5%, 0.05% to 0.45%, 0.05% to 0.4%, 0.05% to 0.35%, 0.05% to 0.3%, 0.05% to 0.25%, 0.05% to 0.2%, 0.1% to 0.4%, 0.1% to 0.45%, 0.1% to 0.4%, 0.1% to 0.35%, 0.1% to 0.3%, 0.1% to 0.25%, 0.1% to 0.2%, or at about 0.15% (w/v).

In some embodiments, the processes described herein comprise obtaining lysine dicarboxylate salt crystals and/or a lysine dicarboxylate salt stream from the fermentation broth via a crystallization step comprising the addition of an equivalent or excess amount of dicarboxylic acid to obtain lysine dicarboxylate salt crystals, which may be subsequently filtered, dried and dissolved in an aqueous solution. Advantageously, the lysine dicarboxylate salt crystals may be dissolved in aqueous solution and then directly employed in a subsequent bioconversion step to enzymatically convert the lysine to cadaverine, without additional lysine purification steps, such as ion exchange or desalting methods conventionally used in the art.

In some embodiments, the processes described herein do not comprise the addition of any further sources of non-essential inorganic ions, thereby minimizing the amount of inorganic ions present in the lysine dicarboxylate salt stream, and thus in downstream intermediates or products derived therefrom. In some embodiments, the processes described herein do not comprise a purification step to remove inorganic and/or non-dicarboxylate anions from the fermentation broth and/or lysine dicarboxylate salt stream.

In some embodiments, the inorganic anions referred to in the processes described herein is or comprise phosphate, sulfate, chloride ions, and/or other non-essential inorganic anions conventionally used in microbial fermentations. The foregoing is in contrast to convention methods for producing lysine by fermentation, which generally employ sulfate ions or chloride anions added to the culture medium as lysine counter anions in order to maintain electrical neutrality, with the main source of sulfate ions being traditionally ammonium sulfate. Furthermore, conventional lysine fermentation methods require purification of lysine from the fermentation broth, most commonly by an ion exchange method. For example, in the case of lysine, after the fermentation broth is made weakly acidic, lysine is typically adsorbed on an ion exchange resin and then desorbed from the resin with ammonium ions. The desorbed lysine is then used “as is” as lysine base, or crystallized as lysine hydrochloride with hydrochloric acid. On the other hand, when conventional methods do not employ a lysine purification step, the sulphate and chloride ions remain as residual components that decrease the purity and quality of the product. Such reduced purity products are commercially inconsequential for the use of lysine such as in animal feed mixtures, but are prohibitive for use as precursors in bio-based nylon syntheses.

Methods such as those described in EP 1182261 and US 2019/0040429 attempt to reduce the presence of chloride or sulfate ions in the fermentation broth by employing carbonate/bicarbonate counter ions. However, such approaches require an additional step of heating at the end of fermentation to distill off the carbonate/bicarbonate ions, thereby increasing the complexity and cost of the lysine production process. In contrast, in some embodiments, the processes described herein do not comprise the use of a carbonate buffering system (e.g., ammonium carbonate and/or ammonium bicarbonate) and/or do not comprise carbonate or carbonate ions as lysine counter anions. Furthermore, in some embodiments, the processes described herein do not comprise or require a distillation step to purify lysine from the lysine dicarboxylate salt stream, thereby streamlining the process.

In some embodiments, any suitable microorganism engineered to produce lysine from a carbon source may be employed. Such engineered microorganisms have been widely utilized in the field for the commercial production of lysine, and any such engineered microorganisms may be suitable for the processes described herein. In some embodiments, the lysine-producing engineered microorganisms described herein may belong to the genus Corynebacterium (e.g., Corynebacterium glutamicum) or Brevibacterium (e.g., Brevibacterium flavum or Brevibacterium lactofermentum). In some embodiments, the lysine-producing engineered microorganisms may be engineered Escherichia coli. In some embodiments, the lysine-producing engineered microorganism may be a fungus or yeast. In some embodiments, the yeast or fungus may belong to the genus Saccharomyces (e.g., Saccharomyces cerevisiae), Pichia (e.g., Pichia kudriavzevii), or Candida (e.g., Candida albicans), or other industrially suitable yeast or fungus species.

In some embodiments, the processes described herein comprise fermenting the microorganism in the presence of a suitable carbon source (e.g., sugars and carbohydrates, such as glucose, sucrose, lactose, fructose, maltose, molasses, starch and cellulose; oils and fats, such as soy oil, sunflower oil, peanut oil and coconut oil: fatty acids, such as palmitic acid, stearic acid and linoleic acid: alcohols, such as glycerol and ethanol; and organic acids, such as acetic acid). In some embodiments, these substances may be used individually or as a mixture.

In some embodiments, the process described herein comprise fermenting the microorganism under culture conditions enabling lysine production while adding a suitable base and/or acid to maintain the pH of the fermentation broth at levels conducive to lysine production by the microorganism. In some embodiments, pH control during fermentation may comprise addition of a suitable base such as ammonium hydroxide to the fermentation broth in sufficient quantities to counter the decreasing pH of the fermentation broth. In some embodiments, the addition of ammonium hydroxide also provides a continual nitrogen source to the microorganism for the lysine production, thereby serving dual purposes during fermentation (pH regulation and nitrogen source). As needed, the pH of the fermentation broth in some embodiments may be lowered/adjusted by the addition of an organic acid, such as a dicarboxylic acid (e.g., adipic acid), and/or an ammonium dicarboxylate (e.g., ammonium adipate). The dicarboxylic acid and/or ammonium dicarboxylate can be selected from succinic acid/ammonium succinate, glutaric acid/ammonium glutarate, adipic acid/ammonium adipate, pimelic acid/ammonium pimelate, suberic acid/ammonium suberate, azelaic acid/ammonium azelate, sebacic acid/ammonium sebacate, undecane dicarboxylic acid/ammonium undecane dicarboxylate, dodecane dicarboxylic acid/ammonium dodecane dicarboxylate, tridecane dicarboxylic acid/ammonium tridecane dicarboxylate, tetradecane dicarboxylic acid/ammonium tetradecane dicarboxylate, pentadecane dicarboxylic acid/ammonium pentadecane dicarboxylate, hexadecane dicarboxylic acid/ammonium hexadecane dicarboxylate, heptadecane dicarboxylic acid/ammonium heptadecane dicarboxylate, octadecane dicarboxylic acid/ammonium octadecane dicarboxylate. Preferably, the dicarboxylic acid and/or ammonium dicarboxylate is selected from adipic acid/ammonium adipate, pimelic acid/ammonium pimelate, suberic acid/ammonium suberate, azelaic acid/ammonium azelate.

In some embodiments, the processes described herein comprise obtaining a lysine dicarboxylate salt stream at the end of fermentation. In some embodiments, the processes described herein may not comprise or require lysine purification steps to remove inorganic and/or non-dicarboxylate ions (e.g., phosphate, sulfate, chloride, carbonate/bicarbonate, or other non-dicarboxylate anions). In some embodiments, the fermentation broth may be subjected to centrifugation and/or filtration steps to remove cells and/or cellular debris, and/or that may be subjected to a crystallization step to obtain lysine dicarboxylate salt crystals, followed by re-dissolving lysine dicarboxylate salt in aqueous solution. In particularly preferable embodiments, the fermentation broth may be subjected to a crystallization step to obtain lysine dicarboxylate salt crystals, followed by re-dissolving lysine dicarboxylate salt in aqueous solution. In some embodiments, the lysine dicarboxylate salt stream so-obtained may advantageously be used directly in subsequent decarboxylation reactions to produce cadaverine and/or cadaverine dicarboxylate salts.

The inventors have discovered that the purification by crystallizing the intermediate lysine salt can allow for an increase in the yield and purity (the amounts of the impurities such as acetic acid, protein, lactic acid, pyruvic acid will be greatly reduced, even reduced to zero in some instances) of the product cadaverine as well as an increased light transmittance and avoidance of yellowing without the need of additional purification steps for lysine such as ion exchange or desalting methods conventionally used in the art, compared to the production method of cadaverine by one-pot fermentation process. In some embodiments, compared to the cadaverine dicarboxylate salt (preferably adipate) produced by one-pot fermentation process, the cadaverine dicarboxylate salt produced by crystallizing the intermediate lysine dicarboxylate salt (preferably adipate) may have one or more of the following characteristics: (1) an increased yield, (2) no impurities such as acetic acid, lactic acid, and pyruvic acid, (3) significantly reduced amount of protein, (4) an increased light transmittance, and (5) no yellowing. In another embodiment, it has been found that the crystallization by using excess amount of dicarboxylic acid can further lead to an increase in the yield and purity of the product cadaverine salt, compared to the crystallization by using equal equivalent of dicarboxylic acid.

In some embodiments, the cadaverine dicarboxylate salt produced by using the crystalline lysine dicarboxylate salt stream has higher yield and purity, compared to the yield and purity of the corresponding inorganic salts of cadaverine (e.g., sulphate and chloride salt) produced from the crystalline lysine sulphate or chloride. In another embodiment, the final product cadaverine dicarboxylate salt can be produced in a higher yield when forming the lysine dicarboxylate salts by using excess amount of dicarboxylic acids, compared to the lysine dicarboxylate salts formed by using equal equivalent of dicarboxylic acid.

In still further embodiments, when forming the lysine dicarboxylate salts described herein, the dicarboxylic acids used may be selected from succinic acid, glutaric acid, adipic acid, pimelic acid, suberic acid, azelaic acid, sebacic acid, undecane dicarboxylic acid, dodecane dicarboxylic acid, tridecane dicarboxylic acid, tetradecane dicarboxylic acid, pentadecane dicarboxylic acid, hexadecane dicarboxylic acid, heptadecane dicarboxylic acid, and octadecane dicarboxylic acid. Particularly, adipic acid, suberic acid, and azelaic acid can be used. In addition, it has been surprisingly found that when the carbon chain length of the used dicarboxylic acid is too long (e.g., dodecane dicarboxylic acid), the yield and purity of the final product cadaverine dicarboxylate salt will decrease.

In some embodiments, the processes described herein comprise obtaining a lysine dicarboxylate salt stream from a fermentation broth having reduced inorganic ion content as compared to a fermentation broth produced via a corresponding process in which an inorganic ion is substituted for the dicarboxylate ion (i.e., in the buffering system employed in the culture medium).

In some embodiments, the process described herein may comprise—prior to a lysine production phase—a growth phase comprising culturing the microorganisms in a growth medium (e.g., formulated to comprise inorganic ions in excess of those considered non-essential for the lysine production phase) in order to optimize or maximize cell growth until a desired level of cell mass is reached, after which the growth media is replaced with the modified culture medium devoid of non-essential inorganic ions described herein. Alternatively, in some embodiments, the process described herein may comprise—prior to a lysine production phase—a growth phase comprising culturing the microorganisms in a minimal growth medium (e.g., formulated to comprise an ammonium dicarboxylate buffering system and to be devoid of non-essential inorganic salts) in order to optimize or maximize cell growth until a desired level of cell mass is reached, wherein the minimal growth medium is the modified culture medium or is replaced with the modified culture medium as described herein.

In some embodiments, the microorganisms may be immobilized to facilitate culture media replacement and/or downstream lysine purification. Examples of suitable cell immobilization methods are discussed and reviewed in Zhu 2007.

In some aspects, described herein is a process for producing a cadaverine dicarboxylate salt having reduced inorganic salt content. The process may comprise or consist essentially of the process steps described herein for the fermentative production of lysine having reduced inorganic ion content, and further comprise subjecting the lysine dicarboxylate salt stream to an enzymatic decarboxylation reaction while maintaining the pH of the solution at a level sufficient for the reaction to occur by adding a dicarboxylic acid to said solution, thereby producing a solution comprising cadaverine dicarboxylate. In various embodiments, any suitable lysine decarboxylase enzyme may be employed for the decarboxylation reaction. In some embodiments, the lysine decarboxylase enzyme employed may be encoded by a nucleic acid sequence at least 60%, 65%, 70%, 75%, 80%, 85%, 90%, 95%, or 99/o identical to SEQ ID NO: 1. In some embodiments, the lysine decarboxylase enzyme employed may comprise an amino acid sequence at least 60%, 65%, 70%, 75%, 80%, 85%, 90%, 95%, or 99% identical to SEQ ID NO: 2.

The lysine decarboxylase addition ratio can be defined as the ratio of adding the weight of lysine decarboxylase (calculated based on the dry basis of the lysine decarboxylase cell) to the weight of lysine in the lysine fermentation broth (based on the molecular weight of lysine dicarboxylate salt). It is common in the art that the lysine decarboxylase addition ratio is relatively high, for example, (1:80)-(1:295). However, when the high activity lysine decarboxylase described herein is used, the lysine decarboxylase addition ratio can be dramatically lowered during the fermentation due to its higher activity. For example, the lysine decarboxylase addition ratio for the high activity lysine decarboxylase described herein can be (1:600)-(1:1000).

In addition, the inventors have discovered that by using the high activity lysine decarboxylase described herein, the yield and purity of the target product cadaverine dicarboxylate salt can also be improved. The high activity lysine decarboxylase described herein is the lysine decarboxylase which is treated by the process comprising the following steps: (1) plasmid containing the kdc gene of the lysine decarboxylase represented by SEQ ID NO: 1 was transformed into E. coli cells; (2) the transformed positive single colony was selected and inoculated into a LB (10 g/L peptone, 5 g/L yeast extract, and 10 g/L sodium chloride) test tube medium, and the medium was inoculated at 30° C. and 180 RPM overnight; (3) flasks containing TB medium (12 g/L peptone, 24 g/L yeast extract, and 4 g/L glycerol) were inoculated at 5% inoculation dosage from the overnight cultures; (4) the flasks were placed into a shaker for incubation, incubation condition: 30° C., 250 RPM, and for about 2 hours; (5) protein expression was induced with 0.2 mM Isopropyl β-D-1-thiogalactopyranoside (IPTG), the induction condition: 30° C., 250 RPM, and for about 4 hours; (6) cells were harvested by centrifugating the fermentation broth and the wet cells after centrifugation were stored at −80° C. In a preferable embodiment, the E. coli is BL21(DE3) E. coli. In a further preferable embodiment, the incubation condition in step (2) was 30° C., 180 RPM, and incubating by shaking for 16 hours. In a further preferable embodiment, the flasks containing TB medium were inoculated at 5% inoculation dosage from 40 mL LB medium fermentation broth cultured overnight. In a further preferable embodiment, the incubation time in step (4) was 2 hours. In a further preferable embodiment, the concentration of the IPTG was 0.2 mM. In a further preferable embodiment, in step (5), the incubation was carried out for 4 more hours at 30° C. while shaking.

U.S. Pat. No. 7,189,543 describes a method for producing cadaverine dicarboxylate involving subjecting a lysine solution to an enzymatic decarboxylation reaction, while maintaining pH within a target range by adding dicarboxylic acid to the solution, and isolating the resulting cadaverine dicarboxylate for example by crystallization. Efforts to replicate the isolation/crystallization methods described in U.S. Pat. No. 7,189,543 did not result in commercially viable cadaverine dicarboxylate salt yields (e.g, see Example 18 resulting in a yield of only 45%). Thus, different methods of crystallization were explored and tested, some of which are described herein. Strikingly, it was found that greatly improved crystallization can be achieved by adding a sufficient volume of an organic solvent to the solution, preferably a cooled organic solvent such as at temperatures below 25° C., 20° C., or 15° C. (e.g., at temperatures ranging from 5° C. to 20° C. or 10° C. to 15° C.). Accordingly, in some embodiments, the cadaverine production process described herein comprises crystallizing cadaverine dicarboxylate salt by adding a sufficient volume of an organic solvent to the solution, wherein addition of the organic solvent increases the amount of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol or isopropanol.

Furthermore, the inventors have surprisingly discovered that when crystallizing the cadaverine dicarboxylate by using alcoholic solvents, the yield and purity of the product obtained by using isopropanol are both higher than those obtained by using common aliphatic low alcohols (e.g., methanol, ethanol, and n-propanol). In an embodiment, the use of isopropanol for crystallizing the cadaverine adipate leads to an increase in the product yield of at least 10%, for example, at least 11%, 12%, 13%, 14%, or 15%, compared to the yield obtained by crystallizing the cadaverine adipate with n-propanol.

In some aspects, described herein is a process for producing a cadaverine dicarboxylate salt having reduced inorganic salt content, the process comprising or consisting essentially of providing an aqueous solution comprising lysine dicarboxylate; subjecting the lysine dicarboxylate to an enzymatic decarboxylation reaction while maintaining the pH of the solution at a level sufficient for the reaction to occur by adding dicarboxylic acid to the solution, thereby producing a solution comprising cadaverine dicarboxylate; and crystallizing the cadaverine dicarboxylate salt by adding a sufficient volume of an organic solvent to the solution, wherein addition of the organic solvent increases the amount of cadaverine dicarboxylate salt crystals recovered (e.g., by at least 20%, 25%, 30%, 35%, or 40%), as compared to a corresponding crystallization process lacking addition of the organic solvent.

In some embodiments, the enzymatic decarboxylation reaction described herein may be performed by subjecting the lysine product or the lysine dicarboxylate to viable or intact cells of a microorganism expressing the lysine decarboxylase. In some embodiments, the viable or intact cells may be immobilized, for example as described in Zhu 2007. In some embodiments, the enzymatic decarboxylation reaction described herein may be performed by subjecting the lysine product or the lysine dicarboxylate to cell lysates of a microorganism expressing the lysine decarboxylase. The use of viable or intact cells instead of cell lysates may facilitate downstream purification of cellular components from the reaction solution.

In some embodiments, the terms “dicarboxylate” and/or “dicarboxylic acid” as used herein refer to dicarboxylates and/or dicarboxylic acids that are useful as precursors or intermediates for the production of nylon salts, nylons, industrially relevant lysine salts, and/or industrially relevant cadaverine salts. In some embodiments, the dicarboxylate and/or dicarboxylic acid may be a dicarboxylate and/or dicarboxylic acid having 4 to 10 carbons (e.g., succinic acid, glutaric acid, adipic acid, pimelic acid, suberic acid, azelaic acid, or sebacic acid). In specific embodiments, the dicarboxylate and/or dicarboxylic acid employed in the processes and products described herein is adipate and/or adipic acid.

In some aspects, described herein are cadaverine dicarboxylate salts produced by a process as described herein. In some aspects, described herein is the use of the cadaverine dicarboxylate salt produced by a process as described herein for the manufacture of a nylon (e.g., nylon 5,6).

### Items

In various aspects, described herein are one or more of the following items:

1. A process for producing lysine dicarboxylate salt crystals having reduced inorganic impurities, the process comprising or consisting essentially of: (a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions; (b) fermenting the microorganism in the presence of the carbon source under culture conditions enabling lysine production, while controlling fermentation broth pH with the addition of ammonium hydroxide to maintain pH in a range conducive to lysine production; and (c) adding a sufficient amount of dicarboxylic acid to the spent fermentation broth to induce formation of lysine dicarboxylate salt crystals, the lysine dicarboxylate salt crystals having reduced inorganic ion content as compared to a lysine inorganic salt obtainable via a corresponding process in which an inorganic anion is substituted for the dicarboxylate anion in the buffering system.
2. The process of item 1, wherein the sufficient amount of dicarboxylic acid added in step (c) corresponds to at least one equivalent with respect to the mols of lysine in the spent fermentation broth.
3. The process of item 1, wherein the sufficient amount of dicarboxylic acid added in step (c) corresponds to an excess equivalent with respect to the mols of lysine in the spent fermentation broth.
4. The process of any one of items 1 to 3, wherein step (b) further comprises supplementing the fermentation broth with an ammonium dicarboxylate solution to maintain total ammonium concentration at a level conducive to lysine production, preferably to maintain total ammonium concentration at 0.05% to 0.5% w/v.
5. The process of any one of items 1 to 4, wherein the spent fermentation broth prior to crystallization has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
6. The process of any one of items 1 to 5, wherein said process: (i) does not comprise the addition of any further sources of non-essential inorganic anions, thereby minimizing the amount of inorganic anions present in the lysine dicarboxylate salt stream; and/or (ii) does not comprise a purification step (e.g., a desalting and/or ion exchange) to remove inorganic ions from the fermentation broth and/or lysine dicarboxylate salt stream.
7. The process of any one of items 1 to 6, wherein the inorganic ion(s) is or comprises phosphate, sulfate, and/or chloride anions.
8. The process of any one of items 1 to 7, wherein said process does not comprise the use of a carbonate buffering system (e.g., ammonium carbonate and/or ammonium bicarbonate) and/or does not comprise carbonate or carbonate anions as lysine counter anions.
9. The process of any one of items 1 to 8, wherein said process does not comprise a distillation step to purify lysine.
10. The process of any one of items 1 to 9, wherein prior to step (a), the microorganisms are cultured in: (i) a growth medium formulated to comprise inorganic salts; or (ii) a growth medium formulated to be devoid of non-essential inorganic salts, until a desired cell mass is reached, after which the growth medium is replaced with said modified culture medium comprising an ammonium dicarboxylate buffering system and being devoid of non-essential inorganic ions.
11. The process of any one of items 1 to 10 wherein the microorganisms engineered to produce lysine are immobilized to facilitate culture media replacement and/or lysine dicarboxylate salt stream processing.
12. The process of any one of items 1 to 11, wherein the microorganisms engineered to produce lysine are bacteria, preferably belonging to the genus Corynebacterium (e.g., Corynebacterium glutamicum) or Brevibacterium (e.g., Brevibacterium flavum or Brevibacterium lactofermentum).
13. A process for producing a cadaverine dicarboxylate salt having reduced organic and inorganic impurities, the process comprising or consisting essentially of steps (a) to (c) as defined in any one of items 1 to 12, the process further comprising: (d) dissolving the isolated lysine dicarboxylate salt crystals from step (c) in aqueous solution and subjecting the lysine to an enzymatic decarboxylation reaction while maintaining the pH of the solution at a level sufficient for said reaction to occur by adding dicarboxylic acid to said solution, thereby producing a solution comprising cadaverine dicarboxylate; and (e) crystallizing cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol.
14. A process for producing a cadaverine dicarboxylate salt having reduced organic and inorganic impurities, the process comprising or consisting essentially of: (i) providing an aqueous solution comprising lysine dicarboxylate; (ii) subjecting the lysine dicarboxylate to an enzymatic decarboxylation reaction while maintaining the pH of the solution at a level sufficient for said reaction to occur by adding dicarboxylic acid to said solution, thereby producing a solution comprising cadaverine dicarboxylate; and (iii) crystallizing the cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol.
15. The process of item 13 or 14, wherein the enzymatic decarboxylation reaction is performed by subjecting the lysine to viable or intact cells of a microorganism expressing lysine decarboxylase.
16. The process of item 15, wherein the viable or intact cells of the microorganism expressing lysine decarboxylase are immobilized.
17. The process of item 15 or 16, wherein the enzymatic decarboxylation reaction is performed by subjecting the lysine to cell lysates of microorganisms expressing lysine decarboxylase.
18. The process of any one of items 15 to 17, wherein the lysine decarboxylase is: the lysine decarboxylase of SEQ ID NO: 2, or a variant thereof having lysine decarboxylase activity comprising an amino acid sequence at least 60%, 65%, 70%, 75%, 80%, 85%, 90%, or 95% identical to SEQ ID NO: 2.
19. The process of any one of items 1 to 18, wherein said dicarboxylate and/or the dicarboxylic acid contains 4 to 18 carbons, 4 to 16 carbons, 4 to 14 carbons, 4 to 12 carbons, 4 to 10 carbons, 4 to 9 carbons, 6 to 10 carbons, or 6 to 9 carbons.
20. The process of any one of items 1 to 19, wherein:
the dicarboxylate is succinate and/or the dicarboxylic acid is succinic acid, thereby producing a lysine dicarboxylate salt which is a lysine succinate salt;
the dicarboxylate is glutarate and/or the dicarboxylic acid is glutaric acid, thereby producing a lysine dicarboxylate salt which is a lysine glutarate salt;
the dicarboxylate is adipate and/or the dicarboxylic acid is adipic acid, thereby producing a lysine dicarboxylate salt which is a lysine adipate salt;
the dicarboxylate is pimelate and/or the dicarboxylic acid is pimelic acid, thereby producing a lysine dicarboxylate salt which is a lysine pimelate salt;
the dicarboxylate is suberate and/or the dicarboxylic acid is suberic acid, thereby producing a lysine dicarboxylate salt which is a lysine suberate salt;
the dicarboxylate is azelate and/or the dicarboxylic acid is azelaic acid, thereby producing a lysine dicarboxylate salt which is a lysine azelate salt; or
the dicarboxylate is sebacate and/or the dicarboxylic acid is sebacic acid, thereby producing a lysine dicarboxylate salt which is a lysine sebacate salt;
the dicarboxylate is undecane dicarboxylate and/or the dicarboxylic acid is undecane dicarboxylic acid, thereby producing a lysine dicarboxylate salt which is a lysine undecane dicarboxylate salt;
the dicarboxylate is dodecane dicarboxylate and/or the dicarboxylic acid is dodecane dicarboxylic acid, thereby producing a lysine dicarboxylate salt which is a lysine dodecane dicarboxylate salt;
the dicarboxylate is tridecane dicarboxylate and/or the dicarboxylic acid is tridecane dicarboxylic acid, thereby producing a lysine dicarboxylate salt which is a lysine tridecane dicarboxylate salt;
the dicarboxylate is tetradecane dicarboxylate and/or the dicarboxylic acid is tetradecane dicarboxylic acid, thereby producing a lysine dicarboxylate salt which is a lysine tetradecane dicarboxylate salt;
the dicarboxylate is pentadecane dicarboxylate and/or the dicarboxylic acid is pentadecane dicarboxylic acid, thereby producing a lysine dicarboxylate salt which is a lysine pentadecane dicarboxylate salt;
the dicarboxylate is hexadecane dicarboxylate and/or the dicarboxylic acid is hexadecane dicarboxylic acid, thereby producing a lysine dicarboxylate salt which is a lysine hexadecane dicarboxylate salt;
the dicarboxylate is heptadecane dicarboxylate and/or the dicarboxylic acid is heptadecane dicarboxylic acid, thereby producing a lysine dicarboxylate salt which is a lysine heptadecane dicarboxylate salt; or
the dicarboxylate is octadecane dicarboxylate and/or the dicarboxylic acid is octadecane dicarboxylic acid, thereby producing a lysine dicarboxylate salt which is a lysine octadecane dicarboxylate salt.
   21. A cadaverine dicarboxylate salt produced by the process of any one of items 13 to 20.
   22. Use of the cadaverine dicarboxylate salt produced by the process of any one of items 13 to 20 for the manufacture of a nylon.
   23. The use of item 22, wherein the cadaverine dicarboxylate salt is cadaverine adipate salt and the nylon is nylon 5,6.
   24. A fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions, wherein the fermentation broth has reduced inorganic ion content as compared to a corresponding fermentation broth employing an inorganic anion instead of a dicarboxylate anion in the buffering system.
   25. The fermentation broth of item 24, which is supplemented with an ammonium dicarboxylate solution to maintain total ammonium concentration at a level conducive to lysine production, preferably to maintain total ammonium concentration at 0.05% to 0.5% w/v.
   26. The fermentation broth of item 24 or 25, wherein the fermentation broth has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
   27. The fermentation broth of item 24 or 26, wherein the microorganisms engineered to produce lysine are as defined in item 11 or 12.
   28. A lysine dicarboxylate salt stream obtained from a lysine fermentation, the lysine dicarboxylate salt stream comprising lysine cations, dicarboxylate anions, and culture medium modified to comprise an ammonium dicarboxylate buffering system and preferably to be devoid of non-essential inorganic ions, wherein the lysine dicarboxylate salt stream has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
   29. The fermentation broth of any one of items 24 to 27, or the lysine dicarboxylate salt stream of item 26, wherein the inorganic ion(s) is or comprises phosphate, sulfate, and/or chloride anions.
   30. The fermentation broth of any one of items 24 to 27 or 29, or the lysine dicarboxylate salt stream of item 28 or 29, wherein said fermentation broth or said lysine dicarboxylate salt stream does not comprise a carbonate buffering system (e.g., ammonium carbonate and/or ammonium bicarbonate) and/or does not comprise carbonate or carbonate anions as lysine counter anions.

### EXAMPLES

### Example 1: General Materials and Methods

The following reference materials are used in the Examples: Recombinant DNA manipulations generally follow methods described by Sambrook et al., 2001. Restriction enzymes, T4 DNA ligase, Rapid DNA Ligation Kit, SanPrep Column DNA Gel Extraction Kit, Plasmid Mini-Prep Kit and agarose are purchased from Sangon Biotech (Shanghai, China). TE buffer contains 10 mM Tris-HCl (pH 8.0) and 1 mM Na₂EDTA (pH 8.0). TAE buffer contains 40 mM Tris-acetate (pH 8.0) and 2 mM Na₂EDTA.

In Example 2, restriction enzyme digests were performed in buffers provided by Sangon Biotech. A typical restriction enzyme digest contains 0.8 μg of DNA in 8 μL of TE, 2 μL of restriction enzyme buffer (10× concentration), 1 μL of bovine serum albumin (0.1 mg/mL), 1 μL of restriction enzyme and 8 μL TE. Reactions are incubated at 37° C. for 1 h and analyzed by agarose gel electrophoresis. The DNA used for cloning experiments was digested and the reaction was terminated by heating at 70° C. for 15 min followed by extraction of the DNA using SanPrep Column DNA Gel Extraction Kit. The concentration of DNA in the sample was determined as follows. An aliquot (10 μL) of DNA was diluted to 1 mL in TE and the absorbance at 260 nm was measured relative to the absorbance of TE. The DNA concentration was calculated based on the fact that the absorbance at 260 nm of 50 μg/mL of double stranded DNA is 1.0.

Agarose gel typically contains 0.7% agarose (w/v) in TAE buffer. Ethidium bromide (0.5 μg/ml) was added to the agarose to allow visualization of DNA fragments under a UV lamp. Agarose gels were run in TAE buffer. The size of the DNA fragments was determined using two sets of 1 kb Plus DNA Ladder obtained from Sangon Biotech.

### Example 2: Cloning, Expression and Activity Testing of Lysine Decarboxylase Expressed in E. coli

An Escherichia coli lysine decarboxylase kdc (2-keto-acid decarboxylase) gene was synthesized and cloned into pET21a (Millipore Sigma, formerly Novagen). The wild-type kdc nucleic acid sequence from E. coli strain BW25113 (E.C. 4.1.1.18) is represented by SEQ ID NO: 1, and amino acid sequence by SEQ ID NO: 2, annotated as lysine decarboxylase.

Plasmid containing the kdc gene represented by SEQ ID NO: 1 was transformed into BL21(DE3) E. coli cells. Empty plasmid pET21a was also transformed as a negative control. For enzyme expression and characterization experiments, flasks containing 40 mL TB were inoculated at 5% from overnight cultures and shaken. Flasks were incubated at 30° C. at 250 rpm shaking for 2 hours, then protein production was induced with 0.2 mM Isopropyl β-D-1-thiogalactopyranoside (IPTG) and incubated for 4 hours at 30° C. at 250 rpm while shaking, then the incubation was finished. Cells were harvested by centrifugation and the wet cells harvested by centrifugation were stored at −80° C.

KDC enzymatic activity was assessed with a pH-based in vitro assay. The enzyme activity was tested using a commercial lysine-HCl salt. Unless otherwise specified, all chemicals were purchased from Sigma-Aldrich Chemical Company (St. Louis, Mo., USA). First, cells were lysed using a benchtop sonicator. The cell lysates were partially clarified by centrifugation (14,000 g for 5 minutes). Protein concentrations of the resulting clarified lysates were measured via Bradford Protein Assay Kit (Sangon Biotech). Lysates were normalized by protein concentration by dilution in 10 mM Tris buffer. The normalized lysates were then diluted 1:5 in 10 mM Tris buffer. 20 μL of lysate was added to each well for the multiple well plate assay. Each condition was performed in triplicate.

The reaction mixture contained 15% lysine-HCl, 0.04% pyridoxal-5′-phosphate (PLP). The pH of each reaction mixture was adjusted to approximately pH 6.5 by the addition of 1M H₂SO₄ and 1N NaOH. Lysate was then added to the reaction mixture while constantly maintaining the pH at 6.5 by the addition of 1M H₂SO₄. The amount of H₂SO₄ used was recorded and used to calculate activities of the enzyme. The assay reaction was completed when the addition of H₂SO₄ was no longer required to maintain pH at 6.5.

### Example 3: Fermentation of Transformed E. coli Overexpressing KDC

For the present Example, the growth medium was prepared as follows: All solutions were prepared in distilled, deionized water. LB medium (1 L) contained Bacto™ tryptone (i.e., enzymatic digest of casein) (10 g), Bacto™ yeast extract (i.e., water-soluble portion of autolyzed yeast cell) (5 g), and NaCl (10 g). LB-glucose medium contains glucose (10 g), MgSO₄ (0.12 g), and thiamine hydrochloride (0.001 g) in 1 L of LB medium. LB-freeze buffer contained K₂HPO₄ (6.3 g), KH₂PO₄ (1.8 g), MgSO₄ (1.0 g), (NH₄)₂SO₄ (0.9 g), sodium citrate dihydrate (0.5 g) and glycerol (44 mL) in 1 L of LB medium. M9 salts (1 L) contains Na₂HPO₄ (6 g), KH₂PO₄ (3 g), NH₄Cl (1 g), and NaCl (0.5 g). M9 minimal medium contained D-glucose (10 g), MgSO₄ (0.12 g), and thiamine hydrochloride (0.001 g) in 1 L of M9 salts. Antibiotics were added where appropriate to the following final concentrations: ampicillin (Ap), 50 μg/mL; chloramphenicol (Cm), 20 μg/mL; kanamycin (Kan), 50 μg/mL; tetracycline (Tc), 12.5 μg/mL. Stock solutions of antibiotics were prepared in water with the exceptions of chloramphenicol, which was prepared in 95% ethanol and tetracycline, which was prepared in 50% aqueous ethanol. Aqueous stock solutions of TPTG were prepared at various concentrations.

The standard fermentation medium (1 L) contained K₂HPO₄ (7.5 g), ammonium iron (III) citrate (0.3 g), citric acid monohydrate (2.1 g), and concentrated H₂SO₄ (1.2 mL). Fermentation medium was adjusted to pH 7.0 by the addition of concentrated NH₄OH before autoclaving. The following supplements were added immediately prior to initiation of the fermentation: D-glucose, MgSO₄ (0.24 g), potassium and trace minerals including (NH₄)₆(Mo₇O₂₄).4H₂O (0.0037 g), ZnSO₄.7H₂O (0.0029 g), H₃BO₃ (0.0247 g), CuSO₄.5H₂O (0.0025 g), and MnCl₂.4H₂O (0.0158 g). IPTG stock solution was added as necessary (e.g., when optical density at 600 nm lied between 15-20) to the indicated final concentration. Glucose feed solution and MgSO₄ (1M) solution were autoclaved separately. Glucose feed solution (650 g/L) was prepared by combining 300 g of glucose and 280 mL of H₂O. Solutions of trace minerals and IPTG were sterilized through 0.22-μm membranes. Antifoam (Sigma 204) was added to the fermentation broth as needed. Typical wet E. coli cell density reached 120 g/L.

### Example 4A: Crystallization of Lysine Sulfate Salt from Lysine Fermentation Supernatant with Excess Sulfuric Acid (1.2 Eq)

Lysine was obtained by fermentation of a commercially available Corynebacterium glutamicum strain engineered to produce lysine from carbohydrates. C. glutamicum was fermented while maintaining tight control over the temperature (generally between 25° C.-37° C., with an optimal temperature at about 30° C.) and pH (between 7.0-7.4). In addition, because of C. glutamicum's high demand for oxygen, the fermentation vessel is aerated during the exponential growth phase.

C. glutamicum was first cultured in a seed culture medium containing: 10 g/L peptone, 5 g/L yeast extract, and 10 g/L sodium chloride introduced into three 1-L Erlenmeyer flasks at an amount of 400 mL, and that was first sterilized by heating at 120° C. for 20 min. After sterilization, the flask was cooled to 30° C., and a C. glutamicum colony grown on an LB plate was inoculated to this medium. The resulting culture was incubated at 30° C. and pH 7.0 for 9 hours with sufficient aeration and stirring.

Following seed culturing, C. glutamicum was then fermented in a medium containing 25 g/L of glucose, 15 g/L ammonium sulfate, 1.5 g/L potassium dihydrogen phosphate, 5 g/L sodium glutamate, 1 g/L sodium chloride, 0.5 g/L yeast extract at pH 7 was introduced in a 15 L stainless steel fermentation tank at an amount of 8 L (this volume will get larger during the fermentation process), and that was first sterilized by heating at 120° C. for 20 min. After the sterilization the tank was cooled to 30° C., then 1 L of the above seed culture was inoculated to the medium, cultured at 30° C. with aeration at 1 vvm and stirred at 800 rpm. The pH was controlled with the addition of 25% ammonium hydroxide solution. When the glucose concentration in the culture was reduced to 0.5-1.0%, the feed solution was added continuously into the medium to maintain sugar levels between 0.5-0.8%. The feed solution contained 110 g/L glucose, 8.9 g/L magnesium sulfate, 5.3 g/L potassium chloride, 4 g/L phosphoric acid, and 1.5 g/L yeast extract at pH 7.0. When the total ammonium level was reduced to 0.15%, ammonium sulfate solution (450 g/L) was supplemented to maintain the total ammonium level at 0.1-0.2%.

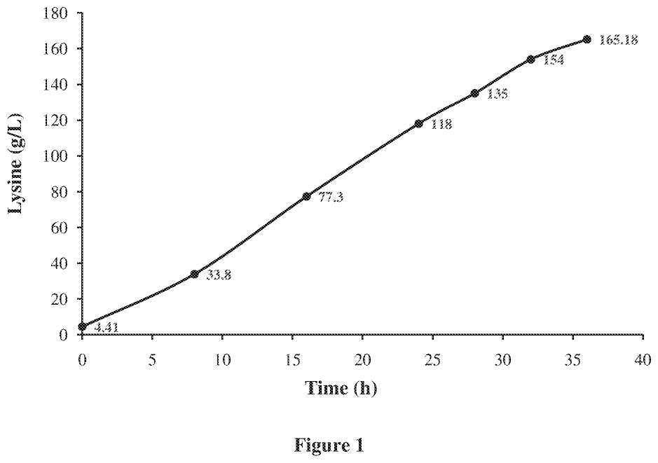

After the fermentation was completed, the total volume of fermentation broth was increased to 10 L by adding glucose, ammonium hydroxide, and ammonium sulfate, and then the cells were separated from the spent medium by centrifugation. The cell-free supernatant was then clarified by micro- and ultra-filtration. Subsequently, this process stream was desalted by passing through ion exchange columns to obtain a solution containing 146 g/L lysine, which was concentrated, and then 1200 g (1.2 equivalents; 1200 g/L) of 98% sulfuric acid was added to crystallize the product lysine sulfate salt. The product was filtered and dried. The purity of lysine-sulfate (i.e., dilysine monosulfate) was then determined by HPLC analysis. The production of lysine by fermentation over time is shown in FIG. 1. The lysine-sulfate salt content was found to be 98.3%, with a yield of 82.6%.

### Example 4B: Crystallization of Lysine Hydrochloride Salt from Lysine Fermentation Supernatant Excess Hydrochloric Acid (1.2 Eq)

Lysine was obtained by fermentation of a commercially available C. glutamicum strain engineered to produce lysine from carbohydrates. C. glutamicum was fermented while maintaining tight control over the temperature (generally between 25° C.-37° C., with an optimal temperature at about 15-30° C.) and pH (between 7.0-7.4). In addition, because of C. glutamicum's high demand for oxygen, the fermentation vessel was aerated during the exponential growth phase. C. glutamicum was first cultured in a seed culture medium containing: 10 g/L peptone, 5 g/L yeast extract, and 10 g/L sodium chloride introduced into three 1-L Erlenmeyer flasks at an amount of 400 mL, and that was first sterilized by heating at 120° C. for 20 min. After sterilization, the flask was cooled to 30° C., and a C. glutamicum colony grown on an LB plate was inoculated to this medium. The resulting culture was incubated at 30° C. and pH 7.0 for 9 hours with sufficient aeration and stirring.

Following seed culturing, C. glutamicum was then fermented in a medium containing 25 g/L of glucose, 15 g/L ammonium chloride, 1.5 g/L potassium dihydrogen phosphate, 5 g/L sodium glutamate, 1 g/L sodium chloride, 0.5 g/L yeast extract at pH 7 was introduced in a 15 L stainless steel fermentation tank at an amount of 8 L (this volume will get larger during the fermentation process), and that was first sterilized by heating at 120° C. for 20 min. After the sterilization the tank was cooled to 30° C., then 1 L of the above seed culture was inoculated to the medium, cultured at 30° C. with aeration at 1 vvm and stirred at 800 rpm. The pH was controlled with the addition of 25% ammonium hydroxide solution. When the glucose concentration in the culture was reduced to 0.5-1.0%, the feed solution was added continuously into the medium to maintain sugar levels between 0.5-0.8%. The feed solution contained 110 g/L glucose, 8.9 g/L magnesium sulfate, 5.3 g/L potassium chloride, 4 g/L phosphoric acid, and 1.5 g/L yeast extract at pH 7.0. When the total ammonium level was reduced to 0.15%, ammonium chloride solution (450 g/L) was supplemented to maintain the total ammonium level at 0.1-0.2%.

After the fermentation was completed, the total volume of fermentation broth was increased to 10 L by adding glucose, ammonium hydroxide, and ammonium chloride, and then the cells were separated from the spent medium by centrifugation. The cell-free supernatant was then clarified by micro- and ultra-filtration. Subsequently, this process stream was desalted by passing through ion exchange columns to obtain a solution containing 146 g/L lysine, which was concentrated, and then 1251 g (125.1 g/L: 1.2 equivalents) of 35% hydrochloric acid was added to crystallize the product lysine hydrochloric salt. The product was filtered and dried. The purity of lysine hydrochloride was then determined by HPLC analysis. The lysine-hydrochloric salt content was found to be 99.1%, with a yield of 87.4%.

### Example 5A: Crystallization of Lysine Adipate Salt from Lysine Fermentation Supernatant with Excess Adipic Acid (1.2 Eq)

Although C. glutamicum can utilize a variety of carbon and nitrogen sources for production, employing a minimal medium having an ammonium adipate buffering system (instead of an ammonium sulfate or ammonium chloride) streamlines downstream processing by reducing inorganic anion content throughout the process and in downstream products.

Following the seed culture as described in Example 4A, C. glutamicum was then fermented in a medium containing 25 g/L of glucose, 10 g/L ammonium adipate, 0.5 g/L potassium dihydrogen phosphate, 5 g/L sodium glutamate, 0.1 g/L sodium chloride, and 0.5 g/L yeast extract at pH 7.0 in a 15 L volume stainless steel fermentation tank at an amount of 8 L (this volume will get larger during the fermentation process), and that was first sterilized by heating at 120° C. for 20 min. Ammonium adipate was prepared by the addition of 3.2 kg adipic acid to a solution of 25% ammonium hydroxide (3 kg) in 7 L water. The resulting solution was stirred at 50° C. for 4 hours. After sterilization of the fermentation medium, the tank is cooled to 30° C., then 1 L of the above seed culture was inoculated to the medium and cultured at 30° C. with aeration of 1 vvm and stirred at 800 rpm. The pH was controlled with the addition of 25% ammonium hydroxide solution. When the glucose concentration in the culture was reduced to 0.5-1.0%, a glucose source was added continuously into the medium to maintain sugar level at between 0.5-0.8%. When the total ammonium level was reduced to 0.15%, ammonium adipate solution (39.7%) was supplemented to maintain the total ammonium level at 0.1-0.2%.

After the fermentation was completed, the total volume of fermentation broth was increased to 10 L by adding glucose, ammonium hydroxide, and ammonium adipate, and then the cells were separated from the spent medium by centrifugation. The cell-free supernatant was then clarified by micro- and ultra-filtration. At this time, the concentration of lysine was 146 g/L, and 1752 g (1.2 equivalents; 175.2 g/L) of adipic acid was added, to crystallize the product lysine adipate salt. The product was filtered and dried. The purity of lysine adipate was then determined by HPLC analysis. The lysine adipate salt content was found to be 99.3%, with a yield of 93.4%.

In this example, the adipate salt ratio was greater than 80%, wherein the adipate salt ratio is calculated as follows: Adipate salt ratio=[(molarity of adipate ions)×2]/[molarity of monocationic lysine ions (Lys+)]×100%. Advantageously, the lysine-adipate salt stream (isolated from cells and cellular debris) is then directly employable as starting material for the bioconversion of lysine adipate to PDA adipate set forth in Examples 13-20, thereby avoiding the need for lysine purification by ion-exchange prior to the bioconversion step.

### Example 5B: Crystallization of Lysine Adipate Salt from Lysine Fermentation Supernatant with 0.6 Eq of Adipic Acid

Although C. glutamicum can utilize a variety of carbon and nitrogen sources for production, employing a minimal medium having an ammonium adipate buffering system (instead of an ammonium sulfate or ammonium chloride) streamlines downstream processing by reducing inorganic anion content throughout the process 30 and in downstream products.

Following the seed culture as described in Example 4, C. glutamicum was then fermented in a medium containing 25 g/L of glucose, 10 g/L ammonium adipate, 0.5 g/L potassium dihydrogen phosphate, 5 g/L sodium glutamate, 0.1 g/L sodium chloride, and 0.5 g/L yeast extract at pH 7.0 in a 15 L volume stainless steel fermentation tank at an amount of 8 L (this volume will get larger during the fermentation process), and that was first sterilized by heating at 120° C. for 20 min. Ammonium adipate was prepared by the addition of 3.2 kg adipic acid to a solution of 25% ammonium hydroxide (3 kg) in 7 L water. The resulting solution was stirred at 50° C. for 4 hours. After sterilization of the fermentation medium, the tank was cooled to 30° C., then 1 L of the above seed culture was inoculated to the medium and cultured at 30° C. with aeration of 1 vvm and stirred at 800 rpm. The pH was controlled with the addition of 25% ammonium hydroxide solution. When the glucose concentration in the culture was reduced to 0.5-1.0%, a glucose source was added continuously into the medium to maintain sugar level at between 0.5-0.8%. When the total ammonium level was reduced to 0.15%, ammonium adipate solution (39.7%) was supplemented to maintain the total ammonium level at 0.1-0.2%.

After the fermentation was completed, the total volume of fermentation broth was increased to 10 L by adding glucose, ammonium hydroxide, and ammonium adipate, and then the cells were separated from the spent medium by centrifugation. The cell-free supernatant was then clarified by micro- and ultra-filtration. At this time, the concentration of lysine was 146 g/L. 876 g (0.6 equivalents; 87.6 g/L) Adipic acid was added to crystallize the product lysine adipate salt. The product was filtered and dried. The purity of lysine adipate was then determined by HPLC analysis, the lysine-adipate salt content was found to be 99.4%, with a yield of 88.5%.

### Example 5C: Lysine Fermentation with Ammonium Adipate but without Crystallizing the Lysine-Adipate Salt

Although C. glutamicum can utilize a variety of carbon and nitrogen sources for production, employing a minimal medium having an ammonium adipate buffering system (instead of an ammonium sulfate or ammonium chloride) streamlines downstream processing by reducing inorganic anion content throughout the process 30 and in downstream products.

Following the seed culture as described in Example 4, C. glutamicum was then fermented in a medium containing 25 g/L of glucose, 10 g/L ammonium adipate, 0.5 g/L potassium dihydrogen phosphate, 5 g/L sodium glutamate, 0.1 g/L sodium chloride, and 0.5 g/L yeast extract at pH 7.0 in a 15 L volume stainless steel fermentation tank at an amount of 8 L, and that was first sterilized by heating at 120° C. for 20 min. Ammonium adipate was prepared by the addition of 3.2 kg adipic acid to a solution of 25% ammonium hydroxide (3 kg) in 7 L water. The resulting solution was stirred at 50° C. for 4 hours. After sterilization of the fermentation medium, the tank was cooled to 30° C., then 1 L of the above seed culture was inoculated to the medium and cultured at 30° C. with aeration of 1 vvm and stirred at 800 rpm. The pH was controlled with the addition of 25% ammonium hydroxide solution. When the glucose concentration in the culture was reduced to 0.5-1.0%, a glucose source was added continuously into the medium to maintain sugar level at between 0.5-0.8%. When the total ammonium level was reduced to 0.15%, ammonium adipate solution (39.7%) was supplemented to maintain the total ammonium level at 0.1-0.2%.

After the fermentation was completed, the total volume of fermentation broth was increased to 10 L by adding glucose, ammonium hydroxide, and ammonium adipate, and then the cells were separated from the spent medium by centrifugation. The cell-free supernatant was then clarified by micro- and ultra-filtration. The concentration of lysine in the supernatant was 146 g/L, and the supernatant was directly used in the next reaction without crystallizing purification.

### Example 6: Conversion of Lysine Hydrochloride to PDA Hydrochloride Using Whole Cells Expressing Lysine Decarboxylase

For the production of pentamethylenediamine (PDA)-HCl, 2 g of lysine decarboxylase-containing wet engineered E. coli (1 g wet cell corresponds to 0.1-0.15 g cell on dry basis) was added to 1 L of a 200 g/L lysine hydrochloride solution containing 0.1 g/L PLP. pH was maintained at 6.5 using HCl. The temperature of the solution was brought to 37° C. Reaction was then started and lasted for 10 hours, while maintaining the pH at 6.5. Lysine content was determined by high performance liquid chromatography (HPLC) at the end of the reaction (<0.5% w/v).

The reaction mixture was passed through a 0.2-micron microfiltration membrane (to remove large particulates such as cells, bacterial fragments and aggregates) and a 10 kDa ultrafiltration membrane (to remove proteins and other soluble macromolecules in culture medium). The filtrate was concentrated to ¼ of the original volume under reduced pressure. A 2× volume of methanol was added to the mixture, and subsequently crystallized at 15° C. The solid was then collected and dried. This white solid product weighed at 174.67 g and was analyzed for PDA-HCl content. PDA-HCl content was found to be 99.3%, with a yield of 91.1%.

### Example 7: Conversion of Lysine Hydrochloride to PDA Hydrochloride Using Lysine Decarboxylase from Cell Lysates

The same method was used for the production of PDA-HCl as in Example 6, however, 2 g of lysates from lysine decarboxylase-containing engineered E. coli cells were added instead of whole cells. For obtaining soluble cell extracts, 2 g of E. coli engineered bacterial cells was added to 10 mL of a phosphate buffer solution (pH 7.0) and stirred well. The cells were then crushed by high-pressure homogenization and then centrifuged to obtain soluble cell extracts. The remaining white solid from the reaction weighed at 172.9 g. PDA-HCl content was found to be 99.5%, with a yield of 90.2%.

### Example 8: PDA Hydrochloride Crystallization with Ethanol

The same method was used for the production of PDA-HCl as in Example 6, however, ethanol was added instead of methanol for crystallization at 15° C. The remaining white solid from the reaction weighed at 177.35 g. PDA-HCl content was found to be 99.2%, with a yield of 92.5%.

### Example 9: PDA Hydrochloride Crystallization with Isopropanol

The same method was used for the production of PDA-HCl as in Example 6, however, 3 times the volume of isopropyl alcohol was added instead of methanol for crystallization at 15° C. The remaining white solid from the reaction weighed at 171.2 g. PDA-HCl content was found to be 99.4%, with a yield of 89.3%.

### Example 10: Increased Lysine Hydrochloride Concentration and Adjusted pH to 7

The same method was used for the production of PDA-HCl as in Example 6, however, the lysine hydrochloride concentration was at 300 g/L with a pH maintained at 7 instead of 200 g/L at a pH of 6.5, 4 g of engineered wet E. coli was added instead of 2 g, 0.15 g/L of PLP instead of 0.1 g/L, and the reaction time was 13 hours instead of 10 hours. The remaining white solid from the reaction weighed at 223.4 g. PDA-HCl content was found to be 99.3%, with a yield of 91.5%.

### Example 11: Conversion of Lysine Sulfate to PDA Sulfate Using Whole Cells Expressing Lysine Decarboxylase

The same method was used for the production of PDA-sulfate as in Example 6, however, the lysine hydrochloride was replaced by lysine sulfate at 200 g/L, the reaction time was 12 hours instead of 10 hours, and crystallization was done with 2 times the volume of ethanol at 15° C. pH was maintained at 6.5 using sulfate. The remaining white solid from the reaction weighed at 201.88 g. PDA-sulfate content was found to be 99.1%, with a yield of 92.1%.

### Example 12: Conversion of Lysine Acetate to PDA Acetate Using Whole Cells Expressing Lysine Decarboxylase

The same method was used for the production of PDA-acetate as in Example 6, however, the lysine hydrochloride was replaced by lysine acetate at 200 g/L, the reaction time was 12 hours instead of 10 hours, and crystallization was done with 2 times the volume of isopropyl alcohol at 15° C. pH was maintained at 6.5 using acetate. The remaining white solid from the reaction weighed at 259.4 g. PDA-acetate content was found to be 99.2%, with a yield of 85.3%.

### Example 13: Conversion of Lysine Adipate to PDA Adipate Using Whole Cells Expressing Lysine Decarboxylase

The same method was used for the production of PDA-adipate as in Example 6, however, the lysine hydrochloride was replaced by the solution of the lysine adipate crystals obtained in Example 5A at 200 g/L, the reaction time was 12 hours instead of 10 hours, and crystallization was done at 15° C. pH was maintained at 6.5 using adipate. The remaining white solid from the reaction weighed at 306.8 g. PDA-adipate content was found to be 99.2%, with a yield of 90.3%.

### Example 14A: Conversion of Lysine Adipate to PDA Adipate Using Immobilized Whole Cells Expressing Lysine Decarboxylase

The same method was used for the production of PDA-adipate as in Example 13, however, 2 g of immobilized lysine decarboxylase-containing E. coli cells were employed instead of whole cells. For cell immobilization, 20 mL of a mixed solution of 8% polyvinyl alcohol and 2.5% sodium alginate was added to 2.5 g of E. coli cells. The solution was mixed well and dripped into a cross-linking agent containing 2% calcium chloride and 3% boric acid from a height of 10 cm using a peristaltic pump. This was then left to solidify for 8 hours, filtered and rinsed repeatedly with distilled water (3-4 times). The immobilized cells were then ready for use at any time and may be reused. The pH of the reaction was maintained at 6.5 using adipate. The remaining white solid from the reaction weighed at 313.56 g. PDA-adipate content was found to be 99.2%, with a yield of 92.3%.

### Example 14B: Conversion of Lysine Adipate to PDA Adipate Using Immobilized Whole Cells Expressing Lysine Decarboxylase

The same method was used for the production of PDA-adipate as in Example 13, however, the lysine hydrochloride was replaced by the solution of the lysine adipate crystals obtained in Example 5B at 200 g/L, and 2 g of immobilized lysine decarboxylase-containing E. coli cells were employed instead of whole cells. For cell immobilization, 20 mL of a mixed solution of 8% polyvinyl alcohol and 2.5% sodium alginate was added to 2.5 g of E. coli cells. The solution was mixed well and dripped into a cross-linking agent containing 25.2% calcium chloride and 3% boric acid from a height of 10 cm using a peristaltic pump. This was then left to solidify for 8 hours, filtered and rinsed repeatedly with distilled water (3-4 times). The immobilized cells were then ready for use at any time and may be reused. The pH of the reaction was maintained at 6.5 using adipate. The remaining white solid from the reaction weighed at 309.82 g. PDA-adipate content was found to be 99.0%, with a yield of 91.3%.

### Example 14C: Conversion of Lysine Adipate to PDA Adipate Using Immobilized Whole Cells Expressing Lysine Decarboxylase

The same method was used for the production of PDA-adipate fermentation reaction solution as in Example 13, however, the lysine hydrochloride was replaced by the supernatant of the lysine-adipate salt fermentation broth obtained in Example 5C, wherein the concentration of the lysine-adipate salt was adjusted to 200 g/L, and 2 g of immobilized lysine decarboxylase-containing E. coli cells were employed instead of whole cells. For cell immobilization, 20 mL of a mixed solution of 8% polyvinyl alcohol and 2.5% sodium alginate was added to 2.5 g of E. coli cells. The solution was mixed well and dripped into a cross-linking agent containing 25.2% calcium chloride and 3% boric acid from a height of 10 cm using a peristaltic pump. This was then left to solidify for 8 hours, filtered and rinsed repeatedly with distilled water (3-4 times). The immobilized cells were then ready for use at any time and may be reused. The pH of the reaction was maintained at 6.5 using adipate. The remaining white solid from the reaction weighed as 299.97 g. PDA-adipate content was found to be 98.6%, with a yield of 88.3%.

### Example 15: Conversion of Lysine Adipate to PDA Adipate Using Lysine Decarboxylase from Cell Lysates

The same method was used for the production of PDA-adipate as in Example 13, however, cell lysates from lysine decarboxylase-containing E. coli cells were employed instead of whole cells. After the reaction, the remaining white solid from the reaction weighed at 308.8 g. PDA-adipate content was found to be 99.1%, with a yield of 90.9%.

### Example 16: PDA-Adipate Crystallization with Ethanol Instead of Methanol

The same method was used for the production of PDA-adipate as in Example 13, however, ethanol was added instead of methanol for crystallization at 15° C. The remaining white solid from the reaction weighed at 311 g. PDA-adipate content was found to be 99.5%, with a yield of 91.5%.

### Example 17: Increasing pH to 7.5 Instead of 6.5

The same method was used for the production of PDA-adipate as in Example 16, however, the pH was controlled at 7.5 with adipic acid instead of 6.5 and the reaction was carried out for 16 hours instead of 12 hours. The remaining white solid from the reaction weighed at 309.2 g. PDA-adipate content was found to be 99.1%, with a yield of 91.0%.

### Example 18: Increasing Lysine-Adipate Concentration from 200 g/L to 400 g/L

The same method was used for the production of PDA-adipate as in Example 16, however, the starting concentration of lysine-adipate was 400 g/L instead of 200 g/L and the reaction was carried out for 18 hours instead of 12 hours. The remaining white solid from the reaction weighed at 615 g. PDA-adipate content was found to be 99.2%, with a yield of 90.5%.

### Example 19A: PDA-Adipate Crystallization with Isopropanol Instead of Methanol

The same method was used for the production of PDA-adipate as in Example 13, however, 3 times the volume of isopropanol was added instead of methanol for crystallization at 15° C. The remaining white solid from the reaction weighed at 312.7 g. PDA-adipate content was found to be 99.1%, with a yield of 92.0%.

### Example 19B: PDA-Adipate Crystallization with n-Propanol Instead of Methanol

The same method was used for the production of PDA-adipate as in Example 13, however, 3 times the volume of n-propanol was added instead of methanol for crystallization at 15° C. The remaining white solid from the reaction weighed at 274.0 g. PDA-adipate content was found to be 99.3%, with a yield of 80.6%.

### Example 20: PDA-Adipate Crystallization by Direct Crystallization in Water

The same method was used for the production of PDA-adipate as in Example 13, however. PDA-adipate crystallization was done directly in water (without the addition of an organic solvent such as methanol, ethanol, isopropanol). The reaction solution was slowly cooled to 4° C. and 1 g of PDA-adipate seed crystals were added, and the crystals were stirred at low speed. The solution was then dried by suction filtration for 10 hours. The remaining white solid from the reaction weighed at 153.9 g. PDA-adipate content was found to be 99.1%, with a yield of only 45.3%.

### Example 21A: Lysine Fermentation with Ammonium Suberate in Combination with Crystallization with Excess Amount of Suberic Add

The same method was used as in Example 5A, however, ammonium suberate buffering system (sebacic acid/ammonium sebacate) was employed instead of ammonium adipate buffering system. After the fermentation was completed and after the separation and filtration, excess amount of suberic acid was added to crystallizing the product lysine suberate. The product was filtered and dried. The purity of lysine suberate was then determined by HPLC analysis. The lysine suberate content was found to be 99.3%, with a yield of 92.5%.

### Example 21B: Lysine Fermentation with Ammonium Azelate in Combination with Crystallization with Excess Amount of Azelaic Add

The same method was used as in Example 5A, however, ammonium azelate buffering system (azelaic acid/ammonium azelate) was employed instead of ammonium adipate buffering system. After the fermentation was completed and after the separation and filtration, excess amount of azelaic acid was added to crystallizing the product lysine azelate. The product was filtered and dried. The purity of lysine azelate was then determined by HPLC analysis. The lysine azelate content was found to be 99.0%, with a yield of 91.2%.

### Example 21C: Lysine Fermentation with Ammonium Dodecane Dicarboxylate in Combination with Crystallization with Excess Amount of Dodecane Dicarboxylic Add

The same method was used as in Example 5A, however, ammonium dodecane dicarboxylate buffering system (dodecane dicarboxylic acid/ammonium dodecane dicarboxylate) was employed instead of ammonium adipate buffering system. After the fermentation was completed and after the separation and filtration, excess amount of dodecane dicarboxylic acid was added to crystallizing the product lysine dodecanedicarboxylate. The product was filtered and dried. The purity of lysine dodecanedicarboxylate was then determined by HPLC analysis. The lysine dodecanedicarboxylate content was found to be 98.6%, with a yield of 88.7%.

### Example 22A: Conversion of Lysine Suberate to PDA Suberate Using Immobilized Whole Cells Expressing Lysine Decarboxylase

The same method was used for the production of PDA-suberate as in Example 13, however, the lysine hydrochloride was replaced by the solution of the lysine suberate crystals obtained in Example 21A at 200 g/L, as well as 2 g of immobilized lysine decarboxylase-containing E. coli cells were employed instead of whole cells. For cell immobilization, 20 mL of a mixed solution of 8% polyvinyl alcohol and 2.5% sodium alginate was added to 2.5 g of E. coli cells. The solution was mixed well and dripped into a cross-linking agent containing 2% calcium chloride and 3% boric acid from a height of 10 cm using a peristaltic pump. This was then left to solidify for 8 hours, filtered and rinsed repeatedly with distilled water (3-4 times). The immobilized cells were then ready for use at any time and may be reused. The pH of the reaction was maintained at 6.5 using suberic acid. The remaining white solid from the reaction weighed at 340.6 g. The PDA suberate content was found to be 99.6%, with a yield of 90.3%.

### Example 22B: Conversion of Lysine Azelate to PDA Azelate Using Immobilized Whole Cells Expressing Lysine Decarboxylase

The same method was used for the production of PDA-azelate as in Example 13, however, the lysine hydrochloride was replaced by the solution of the lysine azelate crystal obtained in Example 21B at 200 g/L, as well as 2 g of immobilized lysine decarboxylase-containing E. coli cells were employed instead of whole cells. For cell immobilization, 20 mL of a mixed solution of 8% polyvinyl alcohol and 2.5% sodium alginate was added to 2.5 g of E. coli cells. The solution was mixed well and dripped into a cross-linking agent containing 2% calcium chloride and 3% boric acid from a height of 10 cm using a peristaltic pump. This was then left to solidify for 8 hours, filtered and rinsed repeatedly with distilled water (3-4 times). The immobilized cells were then ready for use at any time and may be reused. The pH of the reaction was maintained at 6.5 using azelaic acid. The remaining white solid from the reaction weighed at 366.05 g. The PDA azelate content was found to be 99.1%, with a yield of 91.9%.

### Example 22C: Conversion of Lysine Dodecane Dicarboxylate to PDA Dodecane Dicarboxylate Using Immobilized Whole Cells Expressing Lysine Decarboxylase

The same method was used for the production of PDA-dodecane dicarboxylate as in Example 13, however, the lysine hydrochloride was replaced by the solution of the lysine dodecane dicarboxylate crystal obtained in Example 21C at 200 g/L, as well as 2 g of immobilized lysine decarboxylase-containing E. coli cells were employed instead of whole cells. For cell immobilization, 20 mL of a mixed solution of 8% polyvinyl alcohol and 2.5% sodium alginate was added to 2.5 g of E. coli cells. The solution was mixed well and dripped into a cross-linking agent containing 2% calcium chloride and 3% boric acid from a height of 10 cm using a peristaltic pump. This was then left to solidify for 8 hours, filtered and rinsed repeatedly with distilled water (3-4 times). The immobilized cells were then ready for use at any time and may be reused. The pH of the reaction was maintained at 6.5 using dodecane dicarboxylic acid. The remaining white solid from the reaction weighed at 399.46 g. The PDA dodecane dicarboxylate content was found to be 98.1%, with a yield of 87.2%.

### Summary and Conclusions

Lysine Dicarboxylate Salt Crystallization from Spent Lysine Fermentation Supernatant
Examples 4A, 4B, 5A, 5B, 21A, 21B, and 21C compare different approaches for obtaining lysine salt crystals from the cell-free supernatant of a spent lysine fermentation broth. In Examples 4A and 4B, lysine fermentation was carried out in the presence of an ammonium inorganic anion buffering system (containing ammonium sulfate or ammonium chloride), after which the resulting cell-free supernatant was first desalted by passing through ion exchange columns before lysine inorganic salt crystallization (e.g., as lysine sulfate or lysine hydrochloride) was induced by the addition of excess sulfuric acid (Example 4A) or hydrochloric acid (Example 4B). In contrast, the lysine fermentations described in Examples 5A, 5B, 21A, 21B and 21C were carried out in the presence of an ammonium organic anion buffering system (i.e., containing ammonium dicarboxylate), after which lysine organic salt crystallization was induced directly from the cell-free supernatant (i.e., without desalting via an ion exchange column) by the addition of a corresponding dicarboxylic acid—namely: adipic acid (Examples 5A and 5B), suberic acid (Example 21A), azelaic acid (Example 21B), or dodecane dicarboxylic acid (Example 21C). A summary of the yields and purities of the lysine salt crystals obtained is shown below in Table 1.

TABLE 1
Summary of lysine salt crystals obtained in Examples 4A, 4B, 5A, 5B, 21A, 21B, and 21C

| | | | | | Synthesis & |
|---|---|---|---|---|---|
| | Acid used to | Lysine salt crystals | | | crystallization |
| | crystallize lysine salt | obtained | Yield | Purity | method |
| Inorganic | Sulfuric acid | Lysine sulfate | 82.6% | 98.3% | Example 4A |
| acids | (1.2 eq) | | | | |
| | Hydrochloric acid (1.2 | Lysine hydrochloride | 87.4% | 99.1% | Example 4B |
| | eq) | | | | |
| Dicarboxylic | Adipic acid | Lysine adipate | 93.4% | 99.3% | Example 5A |
| acids | (excess) | | | | |
| (C6-C12) | Adipic acid | | 88.5% | 99.4% | Example 5B |
| | (0.6 eq) | | | | |
| | Suberic acid | Lysine suberate | 92.5% | 99.3% | Example 21A |
| | (excess) | | | | |
| | Azelaic acid | Lysine azelate | 91.2% | 99.0% | Example 21B |
| | (excess) | | | | |
| | Dodecane dicarboxylic | Lysine | 88.7% | 98.6% | Example 21C |
| | acid (excess) | dodecanedicarboxylate | | | |

As shown in Table 1, generally higher yields and/or purities of lysine salt crystals were obtained when an ammonium dicarboxylate buffering system was employed in the lysine fermentation and when lysine salt crystallization was induced by addition of a corresponding dicarboxylic acid (Examples 5A, 5B, 21A, 21B, and 21C), as compared to using an ammonium inorganic anion buffering system (Examples 4A and 4B). Strikingly, the yields/purities of the lysine salt crystals obtained using the dicarboxylic acids (Examples 5A, 5B, 21A, 21B, and 21C) were either comparable to, or higher than, the yields/purities obtained using the inorganic anions/acids (Examples 4A and 4B), despite the latter methods including an extra desalting step by passing through ion exchange columns. Furthermore, the results in Examples 5A and 5B suggest that inducing lysine salt crystallization using an equivalent or excess amount of dicarboxylic acid is even more conducive to the formation of crystals, thus resulting in increased yield, compared to using limiting (e.g., 0.6 equivalent) amount of the organic dicarboxylic acid. Interestingly, among the different conditions shown in Table 1, crystallization of lysine dicarboxylate salt with excess adipic acid provided the highest combination of both yield and purity.

### Bioconversion of Lysine Dicarboxylate to PDA Dicarboxylate

Conventional industrial fermentation processes greatly favor streamlined so-called “one-pot” approaches that minimize mid-process purification steps (e.g., of intermediates) to improve overall efficiency and reduce operating costs, with expensive purification steps generally being reserved for the final products. The effects of introducing an additional lysine salt crystallization step on the yield and purity of the final product (PDA adipate salt) was evaluated and compared to a more conventional streamlined-approach lacking the additional lysine salt crystallization step. More specifically, the bioconversions of lysine adipate to PDA adipate in Examples 14A and 14B utilized the lysine adipate salt crystals of Examples 5A and 5B as starting materials, while the bioconversion in Example 14C utilized the lysine adipate fermentation supernatant of Example 5C as starting material. A summary of the yields and purities of the PDA adipate salts produced is shown below in Table 2.

TABLE 2
Comparison of the quality of PDA adipate products obtained by bioconversion
of lysine adipate intermediates produced via different processing methods.

| | | | | | | | | Heat storage result | | |
|---|---|---|---|---|---|---|---|---|---|---|
| | | | | | | | | (250° C., 8 h) | | |
| | | | | Impurity result | | | | | color | |
| Processing | | PDA | | acetic | | lactic | pyruvic | | | dissolved |
| of raw | Synthesis | adipate | | acid | protein | acid | acid | light | | in water |
| material | method | yield | purity | content | content | content | content | transmittance | only storage | after storage |
| A | Example | 92.3% | 99.2% | 0% | 0.08% | 0% | 0% | 99.2% | white solid | colorless |
| | 14A | | | | | | | | | clear solution |
| B | Example | 91.3% | 99.0% | 0% | 0.1% | 0% | 0% | 99.3% | white solid | colorless |
| | 14B | | | | | | | | | clear solution |
| C | Example | 88.3% | 98.6% | 0.23 | 0.41% | 0.06% | 0.11% | 78.4% | pale-yellow | pale-yellow |
| | 14C | | | | | | | | solid | clear solution |

A. crystallization of lysine adipate (excess adipic acid; Example 5A)
B. crystallization of lysine adipate (0.6 equivalent of adipic acid; Example 5B)
C. no crystallization of lysine adipate (Example 5C)

As shown in Table 2, a conventional streamlined production process in which the bioconversion was performed directly on the lysine fermentation supernatant resulted in a PDA adipate yield of 88.3% and an overall purity of 98.6%. Among the impurities detected were substantial amounts of organic impurities such as acidtic acid, protein, lactic acid and pyruvic acid. Upon exposure to heating at 250° C. for 8 h, the negative impact of these organic impurities was visible in the form a yellowing of the PDA adipate product, which was also quantified as a decrease in light transmittance upon dissolution. In contrast, when the bioconversion was performed on resolubilized lysine adipate crystals isolated by adding adipic acid to the lysine fermentation supernatant, the resulting PDA adipate product was not only of higher purity (99.0-99.2%) but was also of significantly higher yield (91.3%-92.3%). The decrease in organic impurities was visible in the form a whiter PDA adipate product, which was also quantified as an increase in light transmittance upon dissolution. Interestingly, use of lysine adipate crystals isolated by adding excess adipic acid to the lysine fermentation supernatant resulted in higher PDA adipate yield and purity, compared to lysine adipate crystals isolated by adding 0.6 equivalent amount of adipic acid. The applicability of the present production process with dicarboxylic acids of longer carbon chain lengths than adipic acid (C6) was also explored in Examples 22A (suberic acid: C8), Example 22B (azelaic acid; C9), and Example 22C (dodecane dicarboxylic acid; C12). A summary of the yields and purities of the PDA dicarboxylate salts produced is shown below in Table 3.

TABLE 3
Yield and purity of PDA dicarboxylate salts obtained by using
organic dicarboxylic acids with different carbon chain lengths

| | | | | | | | | Heat storage result | |
|---|---|---|---|---|---|---|---|---|---|
| | | PDA | | Organic impurities detected | | | | (250° C., 8 h) | |
| | Synthesis | dicarboxylate | | acetic | | lactic | pyruvic | Light | |
| | method | Yield | Parity | acid | protein | acid | acid | transmittance | Color |
| PDA-suberate salt | Example | 90.3% | 99.6% | 0% | 0.08% | 0% | 0% | 99.6% | White |
| | 22A | | | | | | | | solid |
| PDA-azelate salt | Example | 91.9% | 99.1% | 0% | 0.1% | 0% | 0% | 99.1% | White |
| | 22B | | | | | | | | solid |
| PDA-dodecane | Example | 87.2% | 98.1% | 0.08% | 0.15% | 0.01% | 0.02% | 98.5% | White |
| dicarboxylate salt | 22C | | | | | | | | solid |

It can be seen from the results in Table 3, employing dicarboxylic acid of longer carbon chain lengths resulted in strong yields and purities of PDA dicarboxylate salts. However, decreased yield and purity was observed using dicarboxylic acid of twelve carbons.

### Alcohol-Induced Crystallization of PDA Dicarboxylate Salt

The inventors further unexpectedly discovered that, following bioconversion, the yield of the PDA dicarboxylate salt products could be greatly increased by the addition of an alcohol to induce crystallization. While all alcohols tested increased the yield, methanol, ethanol and isopropanol proved to be particularly good at inducing crystallization of a variety of PDA dicarboxylate salts. Interestingly, the highest PDA adipate yields were obtained with isopropanol (yield of 92%; Example 19A), while the alcohol that performed the poorest was n-propanol (yield of 80.6%; Example 19B). Furthermore, strong yields of PDA dicarboxylate crystals were obtained by cooling to about 15° C. instead of to 4° C. in the absence of alcohol (Example 20).

TABLE 4
A summary of the conditions and results from the reactions
of Examples 6-22 for the production of PDA.

| Reaction | | | | | | | | | | | | |
|---|---|---|---|---|---|---|---|---|---|---|---|---|
| Conditions/ | EXAMPLE | | | | | | | | | | | |
| Results | 6 | | 7 | | 8 | | 9 | | 10 | | 11 | |
| Lysine source | Lysine HCl | | Lysine HCl | | Lysine HCl | | Lysine HCl | | Lysine HCl | | Lysine | |
| | (200 g/L) | | (200 g/L) | | (200 g/L) | | (200 g/L) | | (300 g/L) | | sulfate | |
| | | | | | | | | | | | (200 g/L) | |
| Lysine | Whole eng. | | Lysates of eng. | | Whole eng. | | Whole eng. | | Whole eng. | | Whole eng. | |
| decarboxylase | E. coli | | E. coli | | E. coli | | E. coli | | E. coli | | E. coli | |
| source | cells (2 g) | | cells (2 g) | | cells (2 g) | | cells (2 g) | | cells (4 g) | | cells (2 g) | |
| pH | HCl 6.5 | | HCl 6.5 | | HCl 6.5 | | HCl 6.5 | | HCl 7 | | H₂SO₄ 6.5 | |
| PLP | 0.1 | g/L | 0.1 | g/L | 0.1 | g/L | 0.1 | g/L | 0.15 | g/L | 0.1 | g/L |
| Duration | 10 | h | 10 | h | 10 | h | 10 | h | 13 | h | 12 | h |
| Temp. | 37° C. | | 37° C. | | 37° C. | | 37° C. | | 37° C. | | 37° C. | |
| Crystalliz. solvent | Methanol | | Methanol | | Ethanol | | Isopropanol | | Methanol | | Ethanol | |
| | at 10° C. | | at 10° C. | | at 15° C. | | at 15° C. | | at 10° C. | | at 15° C. | |
| Powder weight | 174.67 | g | 172.9 | g | 177.35 | g | 171.2 | g | 223.4 | g | 201.88 | g |

| | | | | | | |
|---|---|---|---|---|---|---|
| PDA-salt content | 99.3% | 99.5% | 99.2% | 99.4% | 99.3% | 99.1% |
| Yield | 91.1% | 90.2% | 92.5% | 89.3% | 91.5% | 92.1% |

| Reaction | | | | | | | | | | |
|---|---|---|---|---|---|---|---|---|---|---|
| Conditions/ | EXAMPLE | | | | | | | | | |
| Results | 12 | | 13 | | 14A | | 14B | | 14C | |
| Lysine source | Lysine | | Lysine | | Lysine | | Lysine | | supernatant | |
| | acetate | | adipate | | adipate | | adipate | | of Lysine | |
| | (200 g/L) | | crystal | | crystal | | crystal | | adipate | |
| | | | obtained | | obtained | | obtained | | obtained | |
| | | | in Ex. 5A | | in Ex. 5A | | in Ex. 5B | | in Ex. 5C | |
| | | | (200 g/L) | | (200 g/L) | | (200 g/L) | | (200 g/L) | |
| Lysine | Whole eng. | | Whole eng. | | Immobil. eng. | | Immobil. eng. | | Immobil. eng. | |
| decarboxylase | E. coli | | E. coli | | E. coli | | E. coli | | E. coli | |
| source | cells (2 g) | | cells (2 g) | | cells (2 g) | | cells (2 g) | | cells (2 g) | |
| pH | Acetic | | Adipic | | Adipic | | Adipic- | | Adipic | |
| | acid 6.5 | | acid 6.5 | | acid 6.5 | | acid 6.5 | | acid 6.5 | |
| PLP | 0.1 | g/L | 0.1 | g/L | 0.1 | g/L | 0.1 | g/L | 0.1 | g/L |
| Duration | 12 | h | 12 | h | 12 | h | 12 | h | 12 | h |
| Temp. | 37° C. | | 37° C. | | 37° C. | | 37° C. | | 37° C. | |
| Crystalliz. solvent | Isopropanol | | Methanol | | Methanol | | Methanol | | Methanol | |
| | at 15° C. | | at 15° C. | | at 15° C. | | at 15° C. | | at 15° C. | |
| Powder weight | 259.4 | g | 306.8 | g | 313.56 | g | 309.82 | g | 299.97 | g |

| | | | | | |
|---|---|---|---|---|---|
| PDA-salt content | 99.2% | 99.2% | 99.2% | 99.2% | 98.6% |
| Yield | 85.3% | 90.3% | 92.3% | 91.3% | 88.3% |

| Reaction | | | | | | | | | | | | |
|---|---|---|---|---|---|---|---|---|---|---|---|---|
| Conditions/ | EXAMPLE | | | | | | | | | | | |
| Results | 15 | | 16 | | 17 | | 18 | | 19A | | 19B | |
| Lysine source | Lysine | | Lysine | | Lysine | | Lysine | | Lysine | | Lysine | |
| | adipate | | adipate | | adipate | | adipate | | adipate | | adipate | |
| | crystal | | crystal | | crystal | | crystal | | crystal | | crystal | |
| | obtained | | obtained | | obtained | | obtained | | obtained | | obtained | |
| | in Ex. 5A | | in Ex. 5A | | in Ex. 5A | | in Ex. 5A | | in Ex. 5A | | in Ex. 5A | |
| | (200 g/L) | | (200 g/L) | | (200 g/L) | | (400 g/L) | | (200 g/L) | | (200 g/L) | |
| Lysine | Lysates of eng. | | Whole eng. | | Whole eng. | | Whole eng. | | Whole eng. | | Whole eng. | |
| decarboxylase | E. coli | | E. coli | | E. coli | | E. coli | | E. coli | | E. coli | |
| source | cells (2 g) | | cells (2 g) | | cells (2 g) | | cells (2 g) | | cells (2 g) | | cells (2 g) | |
| pH | Adipic | | Adipic | | Adipic | | Adipic | | Adipic | | Adipic | |
| | acid 6.5 | | acid 6.5 | | acid 7.5 | | acid 6.5 | | acid 6.5 | | acid 6.5 | |
| PLP | 0.1 | g/L | 0.1 | g/L | 0.1 | g/L | 0.1 | g/L | 0.1 | g/L | 0.1 | g/L |
| Duration | 12 | h | 12 | h | 16 | h | 18 | h | 12 | h | 12 | h |
| Temp. | 37° C. | | 37° C. | | 37° C. | | 37° C. | | 37° C. | | 37° C. | |
| Crystalliz. solvent | Methanol | | Ethanol | | Ethanol | | Ethanol | | Isopropanol | | n-propanol | |
| | at 15° C. | | at 15° C. | | at 15° C. | | at 15° C. | | at 15° C. | | at 15° C. | |
| Powder weight | 308.8 | g | 311 | g | 309.2 | g | 615 | g | 312.7 | g | 274.0 | g |

| | | | | | | |
|---|---|---|---|---|---|---|
| PDA-salt content | 99.1% | 99.5% | 99.1% | 99.2% | 99.1% | 99.3% |
| Yield | 90.9% | 91.5% | 91.0% | 90.5% | 92.0% | 80.6% |

| Reaction | | | | | | | | |
|---|---|---|---|---|---|---|---|---|
| Conditions/ | EXAMPLE | | | | | | | |
| Results | 20 | | 22A | | 22B | | 22C | |
| Lysine source | Lysine | | Lysine | | Lysine | | Lysine | |
| | adipate | | suberate | | azelate | | dodecane | |
| | crystal | | crystal | | crystal | | dicarboxylate | |
| | obtained | | obtained | | obtained | | crystal obtained | |
| | in Ex 5A | | in Ex. 21A | | in Ex. 21B | | in Ex. 21C | |
| | (200 g/L) | | (200 g/L) | | (200 g/L) | | (200 g/L) | |
| Lysine | Whole eng. | | Immobil. eng. | | Immobil. eng. | | Immobil. eng. | |
| decarboxylase | E. coli | | E. coli | | E. coli | | E. coli | |
| source | cells (2 g) | | cells (2 g) | | cells (2 g) | | cells (2 g) | |
| pH | Adipic | | Suberic | | Azelaic | | dodecane | |
| | acid 6.5 | | acid 6.5 | | acid 6.5 | | dicarboxylic | |
| | | | | | | | acid 6.5 | |
| PLP | 0.1 | g/L | 0.1 | g/L | 0.1 | g/L | 0.1 | g/L |
| Duration | 12 | h | 12 | h | 12 | h | 12 | h |
| Temp. | 37° C. | | 37° C. | | 37° C. | | 37° C. | |
| Crystalliz. solvent | None | | Methanol | | Methanol | | Methanol at | |
| | (4° C.) | | at 15° C. | | at 15° C. | | 15° C. | |
| Powder weight | 153.9 | g | 340.6 | g | 366.05 | g | 399.46 | g |

| | | | | |
|---|---|---|---|---|
| PDA-salt content | 99.1% | 99.6% | 99.1% | 98.1% |
| Yield | 45.3% | 90.3% | 91.9% | 87.2% |

In summary, due to the use of a culture medium being modified to comprise an ammonium dicarboxylate buffering system and preferably to be devoid of non-essential inorganic ions, the processes described herein can avoid the problem of producing large amounts of inorganic ions typically produced in conventional biological fermentation process for preparing cadaverine. The reduction in the quantity of inorganic anions used in the culture medium avoids subsequent purification steps necessary to remove inorganic ions, thereby greatly reducing production costs and the damage to the environment associated with disposing of excess inorganic ions. The inventors have surprisingly found that crystallizing the intermediate lysine salt by addition of dicarboxylic acid, and then putting the lysine dicarboxylate crystals back into the production process can allow for a substantial increase in the yield and purity of the final product cadaverine salt, as well as an increased light transmittance and avoidance of yellowing of the product, compared to the production method of cadaverine by a one-pot fermentation process. When using organic dicarboxylic acid for crystallization, the purity and yield of lysine salt crystals obtained by using excess amount of the acid are higher than the corresponding results obtained by using 0.6 equivalent amount of the acid. In addition, the inventors have surprisingly found that by using the high activity lysine decarboxylase described herein, not only can the amount of enzyme used can be significantly reduced and the costs saved, but also the yield and purity of the product cadaverine salt can also be improved. Furthermore, the inventors have surprisingly found that the yield and/or purity of the product cadaverine salt can also be improved by using specific alcoholic solvents to crystalize the final product cadaverine salt. Furthermore, the present invention also preferably uses C6 to C9 organic dicarboxylic acids to form salts, and especially preferably uses adipic acid to form cadaverine salt.

### Embodiments

Embodiment 1. A process for producing a cadaverine dicarboxylate salt having reduced inorganic ion content, the process comprising or consisting essentially of:
(a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions;
(b) fermenting the microorganism in the presence of the carbon source under culture conditions enabling lysine production, while controlling fermentation broth pH with the addition of ammonium hydroxide to maintain pH in a range conducive to lysine production;
(c) obtaining a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system;
(d) subjecting the lysine dicarboxylate salt in the lysine dicarboxylate salt stream to an enzymatic decarboxylation reaction while maintaining the pH of the lysine dicarboxylate salt stream at a level sufficient for said reaction to occur by adding the ammonium dicarboxylate buffering system and adding a high activity lysine decarboxylase in a significantly reduced lysine decarboxylase addition ratio compared to the prior art, thereby producing a solution comprising cadaverine dicarboxylate, wherein the lysine decarboxylase addition ratio is defined as the ratio of the adding weight of lysine decarboxylase calculated based on the dry basis of lysine decarboxylase cell to the weight of lysine in the lysine fermentation broth based on the molecular weight of lysine dicarboxylate salt; and
(e) crystallizing cadaverine dicarboxylate salt by adding a sufficient volume of an organic solvent to the solution.
   Embodiment 2. A process for producing a cadaverine dicarboxylate salt having reduced inorganic ion content, the process comprising or consisting essentially of:
(a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions;
(b) fermenting the microorganism in the presence of the carbon source under culture conditions enabling lysine production, while controlling fermentation broth pH with the addition of ammonium hydroxide to maintain pH in a range conducive to lysine production;
(c) adding equal equivalent or excess dicarboxylic acid to obtain lysine dicarboxylate salt crystals and dissolving the crystals in aqueous solution, to obtain a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system;
(d) subjecting the lysine dicarboxylate salt in the lysine dicarboxylate salt stream to an enzymatic decarboxylation reaction while maintaining the pH of the solution at a level sufficient for said reaction to occur by adding the ammonium dicarboxylate buffering system to said solution, thereby producing a solution comprising cadaverine dicarboxylate; and
(e) crystallizing cadaverine dicarboxylate salt by adding a sufficient volume of an organic solvent to the solution.
   Embodiment 3. The process of Embodiment 1, wherein the high activity lysine decarboxylase is the lysine decarboxylase which is treated by the following procedures:
   (1) plasmid containing the kdc gene of the lysine decarboxylase represented by SEQ ID NO: 1 was transformed into E. coli cells;
   (2) the transformed positive single colony was selected and inoculated into a LB test tube medium, and the medium was inoculated at 30° C. and 180 RPM overnight, wherein the LB test tube medium comprises 10 g/L peptone, 5 g/L yeast extract, and 10 g/L sodium chloride:
   (3) flasks containing TB medium were inoculated at 5% inoculation dosage from the overnight cultures, wherein the TB medium comprises 12 g/L peptone, 24 g/L yeast extract, and 4 g/L glycerol:
   (4) the flasks were placed into a shaker for incubation, incubation condition: 30° C., 250 RPM, and for about 2 hours;
   (5) protein expression was induced with 0.2 mM Isopropyl β-D-1-thiogalactopyranoside IPTG, the induction condition: 30° C., 250 RPM, and for about 4 hours:
   (6) cells were harvested by centrifugating the fermentation broth and the wet cells after centrifugation were stored at −80° C.
   Embodiment 4. The process of Embodiment 3, wherein:
   in procedure (i), the E. coli is BL21(DE3) E. coli; or
   in procedure (ii), the incubation condition was: 30° C., 180 RPM, and incubating by shaking for 16 hours; or
   in procedure (iii), the flasks containing TB medium were inoculated at 5% inoculation dosage from 40 mL LB medium fermentation broth cultured overnight; or
   in procedure (iv), the incubation time was 2 hours; or
   in procedure (v), the concentration of the IPTG was 0.2 mM; or
   in procedure (v), the incubation was carried out for 4 more hours at 30° C. while shaking.
   Embodiment 5. The process of any one of Embodiments 1, 3, or 4, wherein the lysine decarboxylase addition ratio for the high activity lysine decarboxylase is (1:600)-(1:1000).
   Embodiment 6. The process of Embodiment 2, wherein the in step (c), excess dicarboxylic acid is added.
   Embodiment 7. The process of any one of Embodiment 1 to 6, wherein step (b) further comprises supplementing the fermentation broth with an ammonium dicarboxylate solution to maintain total ammonium concentration at a level conducive to lysine production, preferably to maintain total ammonium concentration at 0.05% to 0.5% w/v.
   Embodiment 8. The process of any one of Embodiments 1 to 7, wherein the lysine dicarboxylate salt stream has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
   Embodiment 9. The process of any one of Embodiments 1 to 8, wherein said process:
(i) does not comprise the addition of any further sources of non-essential inorganic anions, thereby minimizing the amount of inorganic anions present in the lysine dicarboxylate salt stream: and/or
(ii) does not comprise a purification step to remove inorganic ions from the fermentation broth and/or lysine dicarboxylate salt stream.
   Embodiment 10. The process of any one of Embodiments 1 to 9, wherein the inorganic ion(s) is or comprises phosphate, sulfate, and/or chloride anions.
   Embodiment 11. The process of any one of Embodiments 1 to 10, wherein said process does not comprise the use of a carbonate buffering system and/or does not comprise carbonate or carbonate anions as lysine counter anions, wherein the carbonate buffering system comprises ammonium carbonate and/or ammonium bicarbonate.
   Embodiment 12. The process of any one of Embodiments 1 to 11, wherein said process does not comprise a distillation step to purify lysine from the lysine dicarboxylate salt stream.
   Embodiment 13. The process of any one of Embodiments 1 to 12, wherein prior to step (a), the microorganisms are cultured in:
(i) a growth medium formulated to comprise inorganic salts; or
(ii) a growth medium formulated to be devoid of non-essential inorganic salts,
   until a desired cell mass is reached, after which the growth medium is replaced with said modified culture medium comprising an ammonium dicarboxylate buffering system and being devoid of non-essential inorganic ions.
   Embodiment 14. The process of any one of Embodiments 1 to 13 wherein the microorganisms engineered to produce lysine are immobilized to facilitate culture media replacement and/or lysine dicarboxylate salt stream processing.
   Embodiment 15. The process of any one of Embodiments 1 to 14, wherein the microorganisms engineered to produce lysine are bacteria.
   Embodiment 16. The process of any one of Embodiments 1 to 15, wherein the step (e) crystallizing the cadaverine dicarboxylate salt by adding a sufficient volume of an organic solvent to the solution includes adding a sufficient volume of an alcohol solvent to the solution to increase the yield of cadaverine dicarboxylate salt crystals recovered by at least 20%, as compared to a corresponding crystallization process lacking addition of the organic solvent.
   Embodiment 17. The process of Embodiment 16, wherein the increase in the yield is at least 25%, or at least 30%, or at least 35%, or at least 40%.
   Embodiment 18. The process of Embodiment 16 or 17, wherein the alcohol solvent is methanol, ethanol, or isopropanol.
   Embodiment 19. The process of Embodiment 18, wherein the alcohol solvent is isopropanol.
   Embodiment 20. The process of any one of Embodiments 1 to 19, wherein the enzymatic decarboxylation reaction is performed by subjecting the lysine in the lysine dicarboxylate salt stream to viable or intact cells of a microorganism expressing lysine decarboxylase.
   Embodiment 21. The process of Embodiment 20, wherein the viable or intact cells of the microorganism expressing lysine decarboxylase are immobilized.
   Embodiment 22. The process of any one of Embodiments 1 to 21, wherein the enzymatic decarboxylation reaction is performed by subjecting the lysine in the lysine dicarboxylate salt stream to cell lysates of microorganisms expressing lysine decarboxylase.
   Embodiment 23. The process of any one of Embodiments 1 to 22, wherein said dicarboxylate and/or the dicarboxylic acid contains 4 to 18 carbons.
   Embodiment 24. The process of any one of Embodiments 1 to 22, wherein said dicarboxylate and/or the dicarboxylic acid contains 6 to 9 carbons.
   Embodiment 25. The process of any one of Embodiments 1 to 22, wherein the dicarboxylate is adipate and/or the dicarboxylic acid is adipic acid, thereby producing a lysine dicarboxylate salt stream which is a lysine adipate salt stream.
   Embodiment 26. The process of Embodiment 9, wherein the purification step is a desalting and/or ion exchange.
   Embodiment 27. The process of Embodiment 15, wherein the bacteria belong to the genus Corynebacterium or Brevibacterium.
   Embodiment 28. The process of Embodiment 27, wherein the bacteria belong to Corynebacterium and are Corynebacterium glutamicum.
   Embodiment 29. The process of Embodiment 27, wherein the bacteria belong to Brevibacterium and are Brevibacterium flavum or Brevibacterium lactofermentum.
   Embodiment 30. A cadaverine dicarboxylate salt produced by the process of any one of Embodiments 1 to 29.
   Embodiment 31. Use of the cadaverine dicarboxylate salt produced by the process of any one of Embodiments 1 to 29 for the manufacture of a nylon.
   Embodiment 32. The use of Embodiment 31, wherein the cadaverine dicarboxylate salt is cadaverine adipate salt and the nylon is nylon 5,6.
   Embodiment 33. A process for producing lysine dicarboxylate salt crystals having reduced inorganic impurities, the process comprising or consisting essentially of:
(a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions;
(b) fermenting the microorganism in the presence of the carbon source under culture conditions enabling lysine production, while controlling fermentation broth pH with the addition of ammonium hydroxide to maintain pH in a range conducive to lysine production; and
(c) adding a sufficient amount of dicarboxylic acid to the spent fermentation broth to induce formation of lysine dicarboxylate salt crystals, the lysine dicarboxylate salt crystals having reduced inorganic ion content as compared to a lysine inorganic salt obtainable via a corresponding process in which an inorganic anion is substituted for the dicarboxylate anion in the buffering system.
   Embodiment 34. The process of Embodiment 33, wherein the sufficient amount of dicarboxylic acid added in step (c) corresponds to at least one equivalent with respect to the mols of lysine in the spent fermentation broth.
   Embodiment 35. The process of Embodiment 33, wherein the sufficient amount of dicarboxylic acid added in step (c) corresponds to an excess equivalent with respect to the mols of lysine in the spent fermentation broth.
   Embodiment 36. The process of any one of Embodiments 33 to 35, wherein step (b) further comprises supplementing the fermentation broth with an ammonium dicarboxylate solution to maintain total ammonium concentration at a level conducive to lysine production, preferably to maintain total ammonium concentration at 0.05% to 0.5% w/v.
   Embodiment 37. The process of any one of Embodiments 33 to 36, wherein the spent fermentation broth prior to crystallization has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
   Embodiment 38. The process of any one of Embodiments 33 to 37, wherein said process:
(i) does not comprise the addition of any further sources of non-essential inorganic anions, thereby minimizing the amount of inorganic anions present in the lysine dicarboxylate salt stream: and/or
(ii) does not comprise a purification step (e.g., a desalting and/or ion exchange) to remove inorganic ions from the fermentation broth and/or lysine dicarboxylate salt stream.
   Embodiment 39. The process of any one of Embodiments 33 to 38, wherein the inorganic ion(s) is or comprises phosphate, sulfate, and/or chloride anions.
   Embodiment 40. The process of any one of Embodiments 33 to 39, wherein said process does not comprise the use of a carbonate buffering system (e.g., ammonium carbonate and/or ammonium bicarbonate) and/or does not comprise carbonate or carbonate anions as lysine counter anions.
   Embodiment 41. The process of any one of Embodiments 33 to 40, wherein said process does not comprise a distillation step to purify lysine.
   Embodiment 42. The process of any one of Embodiments 33 to 41, wherein prior to step (a), the microorganisms are cultured in:
(i) a growth medium formulated to comprise inorganic salts, or
(ii) a growth medium formulated to be devoid of non-essential inorganic salts,
   until a desired cell mass is reached, after which the growth medium is replaced with said modified culture medium comprising an ammonium dicarboxylate buffering system and being devoid of non-essential inorganic ions.
   Embodiment 43. The process of any one of Embodiments 33 to 42 wherein the microorganisms engineered to produce lysine are immobilized to facilitate culture media replacement and/or lysine dicarboxylate salt stream processing.
   Embodiment 44. The process of any one of Embodiments 33 to 43, wherein the microorganisms engineered to produce lysine are bacteria, preferably belonging to the genus Corynebacterium (e.g., Corynebacterium glutamicum) or Brevibacterium (e.g., Brevibacterium flavum or Brevibacterium lactofermentum).
   Embodiment 45. A process for producing a cadaverine dicarboxylate salt having reduced organic and inorganic impurities, the process comprising or consisting essentially of steps (a) to (c) as defined in any one of Embodiments 33 to 44, the process further comprising:
(d) dissolving the isolated lysine dicarboxylate salt crystals from step (c) in aqueous solution and subjecting the lysine to an enzymatic decarboxylation reaction while maintaining the pH of the solution at a level sufficient for said reaction to occur by adding dicarboxylic acid to said solution, thereby producing a solution comprising cadaverine dicarboxylate, and
(e) crystallizing cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol.
   Embodiment 46. A process for producing a cadaverine dicarboxylate salt having reduced organic and inorganic impurities, the process comprising or consisting essentially of:
(i) providing an aqueous solution comprising lysine dicarboxylate;
(ii) subjecting the lysine dicarboxylate to an enzymatic decarboxylation reaction while maintaining the pH of the solution at a level sufficient for said reaction to occur by adding dicarboxylic acid to said solution, thereby producing a solution comprising cadaverine dicarboxylate; and
(iii) crystallizing the cadaverine dicarboxylate salt by adding a sufficient volume of an organic solvent to the solution to increase the yield of cadaverine dicarboxylate salt crystals recovered (e.g., by at least 20%, 25%, 30%, 35%, or 40%), as compared to a corresponding crystallization process lacking addition of the organic solvent, the organic solvent being preferably an alcohol such as methanol, ethanol, or isopropanol.
   Embodiment 47. The process of Embodiment 45 or 46, wherein the enzymatic decarboxylation reaction is performed by subjecting the lysine to viable or intact cells of a microorganism expressing lysine decarboxylase.
   Embodiment 48. The process of Embodiment 47, wherein the viable or intact cells of the microorganism expressing lysine decarboxylase are immobilized.
   Embodiment 49. The process of Embodiment 47 or 48, wherein the enzymatic decarboxylation reaction is performed by subjecting the lysine to cell lysates of microorganisms expressing lysine decarboxylase.
   Embodiment 50. The process of any one of Embodiments 47 to 49, wherein the lysine decarboxylase is: the lysine decarboxylase of SEQ ID NO: 2, or a variant thereof having lysine decarboxylase activity comprising an amino acid sequence at least 60%, 65%, 70%, 75%, 80%, 85%, 90%, or 95% identical to SEQ ID NO: 2.
   Embodiment 51. The process of any one of Embodiments 33 to 50, wherein said dicarboxylate and/or the dicarboxylic acid contains 4 to 18 carbons, 4 to 16 carbons, 4 to 14 carbons, 4 to 12 carbons, 4 to 10 carbons, 4 to 9 carbons, 6 to 10 carbons, or 6 to 9 carbons.
   Embodiment 52. The process of any one of Embodiments 33 to 51, wherein:
the dicarboxylate is succinate and/or the dicarboxylic acid is succinic acid, thereby producing a lysine dicarboxylate salt which is a lysine succinate salt;
the dicarboxylate is glutarate and/or the dicarboxylic acid is glutaric acid, thereby producing a lysine dicarboxylate salt which is a lysine glutarate salt;
the dicarboxylate is adipate and/or the dicarboxylic acid is adipic acid, thereby producing a lysine dicarboxylate salt which is a lysine adipate salt;
the dicarboxylate is pimelate and/or the dicarboxylic acid is pimelic acid, thereby producing a lysine dicarboxylate salt which is a lysine pimelate salt;
the dicarboxylate is suberate and/or the dicarboxylic acid is suberic acid, thereby producing a lysine dicarboxylate salt which is a lysine suberate salt;
the dicarboxylate is azelate and/or the dicarboxylic acid is azelaic acid, thereby producing a lysine dicarboxylate salt which is a lysine azelate salt; or
the dicarboxylate is sebacate and/or the dicarboxylic acid is sebacic acid, thereby producing a lysine dicarboxylate salt which is a lysine sebacate salt;
the dicarboxylate is undecane dicarboxylate and/or the dicarboxylic acid is undecane dicarboxylic acid, thereby producing a lysine dicarboxylate salt which is a lysine undecane dicarboxylate salt;
the dicarboxylate is dodecane dicarboxylate and/or the dicarboxylic acid is dodecane dicarboxylic acid, thereby producing a lysine dicarboxylate salt which is a lysine dodecane dicarboxylate salt;
the dicarboxylate is tridecane dicarboxylate and/or the dicarboxylic acid is tridecane dicarboxylic acid, thereby producing a lysine dicarboxylate salt which is a lysine tridecane dicarboxylate salt;
the dicarboxylate is tetradecane dicarboxylate and/or the dicarboxylic acid is tetradecane dicarboxylic acid, thereby producing a lysine dicarboxylate salt which is a lysine tetradecane dicarboxylate salt;
the dicarboxylate is pentadecane dicarboxylate and/or the dicarboxylic acid is pentadecane dicarboxylic acid, thereby producing a lysine dicarboxylate salt which is a lysine pentadecane dicarboxylate salt;
the dicarboxylate is hexadecane dicarboxylate and/or the dicarboxylic acid is hexadecane dicarboxylic acid, thereby producing a lysine dicarboxylate salt which is a lysine hexadecane dicarboxylate salt;
the dicarboxylate is heptadecane dicarboxylate and/or the dicarboxylic acid is heptadecane dicarboxylic acid, thereby producing a lysine dicarboxylate salt which is a lysine heptadecane dicarboxylate salt; or
the dicarboxylate is octadecane dicarboxylate and/or the dicarboxylic acid is octadecane dicarboxylic acid, thereby producing a lysine dicarboxylate salt which is a lysine octadecane dicarboxylate salt.
   Embodiment 53. A cadaverine dicarboxylate salt produced by the process of any one of Embodiments 45 to 52.
   Embodiment 54. Use of the cadaverine dicarboxylate salt produced by the process of any one of Embodiments 45 to 52 for the manufacture of a nylon.
   Embodiment 55. The use of Embodiment 54, wherein the cadaverine dicarboxylate salt is cadaverine adipate salt and the nylon is nylon 5,6.
   Embodiment 56. A fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions, wherein the fermentation broth has reduced inorganic ion content as compared to a corresponding fermentation broth employing an inorganic anion instead of a dicarboxylate anion in the buffering system.
   Embodiment 57. The fermentation broth of Embodiment 56, which is supplemented with an ammonium dicarboxylate solution to maintain total ammonium concentration at a level conducive to lysine production, preferably to maintain total ammonium concentration at 0.05% to 0.5% w/v.
   Embodiment 58. The fermentation broth of Embodiment 56 or 57, wherein the fermentation broth has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
   Embodiment 59. The fermentation broth of Embodiment 56 or 58, wherein the microorganisms engineered to produce lysine are as defined in Embodiment 43 or 44.
   Embodiment 60. A lysine dicarboxylate salt stream obtained from a lysine fermentation, the lysine dicarboxylate salt stream comprising lysine cations, dicarboxylate anions, and culture medium modified to comprise an ammonium dicarboxylate buffering system and preferably to be devoid of non-essential inorganic ions, wherein the lysine dicarboxylate salt stream has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.
   Embodiment 61. The fermentation broth of any one of Embodiments 56 to 59, or the lysine dicarboxylate salt stream of Embodiment 26, wherein the inorganic ion(s) is or comprises phosphate, sulfate, and/or chloride anions.
   Embodiment 62. The fermentation broth of any one of Embodiments 56 to 59 or 61, or the lysine dicarboxylate salt stream of Embodiment 28 or 29, wherein said fermentation broth or said lysine dicarboxylate salt stream does not comprise a carbonate buffering system (e.g., ammonium carbonate and/or ammonium bicarbonate) and/or does not comprise carbonate or carbonate anions as lysine counter anions.

### REFERENCES

Kobayashi et al., EP 118226.
Nishi et al., U.S. Pat. No. 7,189,543.
Sambrook et al. (2001). Molecular Cloning: A Laboratory Manual, Third Edition, Sambrook and Russell, Cold Spring Harbor Laboratory Press, 3^{rd} Edition.
Suzuki et al., US 2019/0040429.
Tsuge et al. (2016). Engineering cell factories for producing building block chemicals for bio-polymer synthesis. Microbial Cell Factories, 15: 19, 1-12.
Zhu (2007). Chapter 14. Immobilized Cell Fermentation for Production of Chemicals and Fuels. Bioprocessing for Value-Added Products from Renewable Resources: Shang-Tian Yang (Editor); pp. 393-396.

1. A process for producing a cadaverine dicarboxylate salt having reduced inorganic ion content, the process comprising or consisting essentially of:
(a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions;
(b) fermenting the microorganism in the presence of the carbon source under culture conditions enabling lysine production, while controlling fermentation broth pH with the addition of ammonium hydroxide to maintain pH in a range conducive to lysine production;
(c) obtaining a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system;
(d) subjecting the lysine dicarboxylate salt in the lysine dicarboxylate salt stream to an enzymatic decarboxylation reaction while maintaining the pH of the lysine dicarboxylate salt stream at a level sufficient for said reaction to occur by adding the ammonium dicarboxylate buffering system and adding a high activity lysine decarboxylase in a significantly reduced lysine decarboxylase addition ratio compared to the prior art, thereby producing a solution comprising cadaverine dicarboxylate, wherein the lysine decarboxylase addition ratio is defined as the ratio of the adding weight of lysine decarboxylase calculated based on the dry basis of lysine decarboxylase cell to the weight of lysine in the lysine fermentation broth based on the molecular weight of lysine dicarboxylate salt; and
(e) crystallizing cadaverine dicarboxylate salt by adding a sufficient volume of an organic solvent to the solution.

2. A process for producing a cadaverine dicarboxylate salt having reduced inorganic ion content, the process comprising or consisting essentially of:
(a) providing a fermentation broth comprising microorganisms immersed in a modified culture medium, the microorganisms being engineered to produce lysine from a carbon source and the culture medium being modified to comprise an ammonium dicarboxylate buffering system and preferably to be devoid of non-essential inorganic ions;
(b) fermenting the microorganism in the presence of the carbon source under culture conditions enabling lysine production, while controlling fermentation broth pH with the addition of ammonium hydroxide to maintain pH in a range conducive to lysine production;
(c) adding equal equivalent or excess dicarboxylic acid to obtain lysine dicarboxylate salt crystals and dissolving the crystals in aqueous solution, to obtain a lysine dicarboxylate salt stream from the fermentation broth, the lysine dicarboxylate salt stream having reduced inorganic ion content as compared to a lysine inorganic salt stream obtainable via a corresponding process in which an inorganic anion is substituted for the dicarboxylate anion in the buffering system;
(d) subjecting the lysine dicarboxylate salt in the lysine dicarboxylate salt stream to an enzymatic decarboxylation reaction while maintaining the pH of the solution at a level sufficient for said reaction to occur by adding the ammonium dicarboxylate buffering system to said solution, thereby producing a solution comprising cadaverine dicarboxylate; and
(e) crystallizing cadaverine dicarboxylate salt by adding a sufficient volume of an organic solvent to the solution.

3. The process of claim 1, wherein the high activity lysine decarboxylase is the lysine decarboxylase which is treated by the following procedures:
(1) plasmid containing the kdc gene of the lysine decarboxylase represented by SEQ ID NO: 1 was transformed into E. coli cells;
(2) the transformed positive single colony was selected and inoculated into a LB test tube medium, and the medium was inoculated at 30° C. and 180 RPM overnight, wherein the LB test tube medium comprises 10 g/L peptone, 5 g/L yeast extract, and 10 g/L sodium chloride;
(3) flasks containing TB medium were inoculated at 5% inoculation dosage from the overnight cultures, wherein the TB medium comprises 12 g/L peptone, 24 g/L yeast extract, and 4 g/L glycerol;
(4) the flasks were placed into a shaker for incubation, incubation condition: 30° C., 250 RPM, and for about 2 hours;
(5) protein expression was induced with 0.2 mM Isopropyl β-D-1-thiogalactopyranoside IPTG, the induction condition: 30° C., 250 RPM, and for about 4 hours;
(6) cells were harvested by centrifugating the fermentation broth and the wet cells after centrifugation were stored at −80° C.

4. The process of claim 3, wherein:
in procedure (i), the E. coli is BL21(DE3) E. coli; or
in procedure (ii), the incubation condition was: 30° C., 180 RPM, and incubating by shaking for 16 hours; or
in procedure (iii), the flasks containing TB medium were inoculated at 5% inoculation dosage from 40 mL LB medium fermentation broth cultured overnight; or
in procedure (iv), the incubation time was 2 hours; or
in procedure (v), the concentration of the IPTG was 0.2 mM; or
in procedure (v), the incubation was carried out for 4 more hours at 30° C. while shaking.

5. The process of any one of claims 1, 3, or 4, wherein the lysine decarboxylase addition ratio for 5 the high activity lysine decarboxylase is (1:600)-(1:1000).

6. The process of claim 2, wherein the in step (c), excess dicarboxylic acid is added.

7. The process of any one of claims 1 to 6, wherein step (b) further comprises supplementing the fermentation broth with an ammonium dicarboxylate solution to maintain total ammonium concentration at a level conducive to lysine production, preferably to maintain total ammonium concentration at 0.05% to 0.5% w/v.

8. The process of any one of claims 1 to 7, wherein the lysine dicarboxylate salt stream has a Dicarboxylate Salt Ratio (DSR) of at least 50%, 55%, 60%, 65%, 70%, 75%, or 80%, as calculated using the following formula: DSR=[(molarity of dicarboxylate ions)×2]/[molarity of monocationic lysine (Lys+) ions]×100%.

9. The process of any one of claims 1 to 8, wherein said process:
(i) does not comprise the addition of any further sources of non-essential inorganic anions, thereby minimizing the amount of inorganic anions present in the lysine dicarboxylate salt stream; and/or
(ii) does not comprise a purification step to remove inorganic ions from the fermentation broth and/or lysine dicarboxylate salt stream.

10. The process of any one of claims 1 to 9, wherein the inorganic ion(s) is or comprises phosphate, sulfate, and/or chloride anions.

11. The process of any one of claims 1 to 10, wherein said process does not comprise the use of a carbonate buffering system and/or does not comprise carbonate or carbonate anions as lysine counter anions, wherein the carbonate buffering system comprises ammonium carbonate and/or ammonium bicarbonate.

12. The process of any one of claims 1 to 11, wherein said process does not comprise a distillation step to purify lysine from the lysine dicarboxylate salt stream.

13. The process of any one of claims 1 to 12, wherein prior to step (a), the microorganisms are cultured in:
(i) a growth medium formulated to comprise inorganic salts; or
(ii) a growth medium formulated to be devoid of non-essential inorganic salts,
until a desired cell mass is reached, after which the growth medium is replaced with said modified culture medium comprising an ammonium dicarboxylate buffering system and being devoid of non-essential inorganic ions.

14. The process of any one of claims 1 to 13 wherein the microorganisms engineered to produce lysine are immobilized to facilitate culture media replacement and/or lysine dicarboxylate salt stream processing.

15. The process of any one of claims 1 to 14, wherein the microorganisms engineered to produce lysine are bacteria.

16. The process of any one of claims 1 to 15, wherein the step (e) crystallizing the cadaverine dicarboxylate salt by adding a sufficient volume of an organic solvent to the solution includes adding a sufficient volume of an alcohol solvent to the solution to increase the yield of cadaverine dicarboxylate salt crystals recovered by at least 20%, as compared to a corresponding crystallization process lacking addition of the organic solvent.

17. The process of claim 16, wherein the increase in the yield is at least 25%, or at least 30%, or at least 35%, or at least 40%.

18. The process of claim 16 or 17, wherein the alcohol solvent is methanol, ethanol, or isopropanol.

19. The process of claim 18, wherein the alcohol solvent is isopropanol.

20. The process of any one of claims 1 to 19, wherein the enzymatic decarboxylation reaction is performed by subjecting the lysine in the lysine dicarboxylate salt stream to viable or intact cells of a microorganism expressing lysine decarboxylase.

21. The process of claim 20, wherein the viable or intact cells of the microorganism expressing lysine decarboxylase are immobilized.

22. The process of any one of claims 1 to 21, wherein the enzymatic decarboxylation reaction is performed by subjecting the lysine in the lysine dicarboxylate salt stream to cell lysates of microorganisms expressing lysine decarboxylase.

23. The process of any one of claims 1 to 22, wherein said dicarboxylate and/or the dicarboxylic acid contains 4 to 18 carbons.

24. The process of any one of claims 1 to 22, wherein said dicarboxylate and/or the dicarboxylic acid contains 6 to 9 carbons.

25. The process of any one of claims 1 to 22, wherein the dicarboxylate is adipate and/or the dicarboxylic acid is adipic acid, thereby producing a lysine dicarboxylate salt stream which is a lysine adipate salt stream.

26. The process of claim 9, wherein the purification step is a desalting and/or ion exchange.

27. The process of claim 15, wherein the bacteria belong to the genus Corynebacterium or Brevibacterium.

28. The process of claim 27, wherein the bacteria belong to Corynebacterium and are Corynebacterium glutamicum.

29. The process of claim 27, wherein the bacteria belong to Brevibacterium and are Brevibacterium flavum or Brevibacterium lactofermentum.

30. A cadaverine dicarboxylate salt produced by the process of any one of claims 1 to 29.

31. Use of the cadaverine dicarboxylate salt produced by the process of any one of claims 1 to 29 for the manufacture of a nylon.

32. The use of claim 31, wherein the cadaverine dicarboxylate salt is cadaverine adipate salt and the nylon is nylon 5,6.
